SEQUENCE LISTING (86 sequences)

SEQ ID NO: 1 - protein, 5 aa, artificial sequences
  /mol_type=“protein” /note=“CDR-H1” /organism=“artificial sequences”
DTYIH

SEQ ID NO: 2 - protein, 17 aa, artificial sequences
  /mol_type=“protein” /note=“CDR-H2” /organism=“artificial sequences”
RIYPTNGYTRYADSVKG

SEQ ID NO: 3 - protein, 11 aa, artificial sequences
  /mol_type=“protein” /note=“CDR-H3” /organism=“artificial sequences”
WGGDGFYAMDY

SEQ ID NO: 4 - protein, 11 aa, artificial sequences
  /mol_type=“protein” /note=“CDR-L1” /organism=“artificial sequences”
RASQDVNTAVA

SEQ ID NO: 5 - protein, 7 aa, artificial sequences
  /mol_type=“protein” /note=“CDR-L2” /organism=“artificial sequences”
SASFLYS

SEQ ID NO: 6 - protein, 9 aa, artificial sequences
  /mol_type=“protein” /note=“CDR-L3” /organism=“artificial sequences”
QQHYTTPPT

SEQ ID NO: 7 - protein, 119 aa, artificial sequences
  /mol_type=“protein” /note=“heavy chain variable region” /organism=“artificial sequences”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 8 - protein, 108 aa, artificial sequences
  /mol_type=“protein” /note=“light chain variable region” /organism=“artificial sequences”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 9 - protein, 450 aa, artificial sequences
  /mol_type=“protein” /note=“heavy chain” /organism=“artificial sequences”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 10 - protein, 214 aa, artificial sequences
  /mol_type=“protein” /note=“light chain” /organism=“artificial sequences”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, Artificial Sequence
  Description of Artificial Sequence Synthetic Peptide
DAWMD

SEQ ID NO: 12 - protein, 5 aa, Artificial Sequence
  Description of Artificial Sequence Synthetic Peptide
NYWMN

SEQ ID NO: 13 - protein, 19 aa, Artificial Sequence
  Description of Artificial Sequence Synthetic Peptide
EIRSKANNHATYYAESVKG

SEQ ID NO: 14 - protein, 19 aa, Artificial Sequence
  Description of Artificial Sequence Synthetic Peptide
EIRLKSNNYTTHYAESVKG

SEQ ID NO: 15 - protein, 7 aa, Artificial Sequence
  Description of Artificial Sequence Synthetic Peptide
GGYGFDY

SEQ ID NO: 16 - protein, 6 aa, Artificial Sequence
  Description of Artificial Sequence Synthetic Peptide
HYYFDY

SEQ ID NO: 17 - protein, 16 aa, Artificial Sequence
  Description of Artificial Sequence Synthetic Peptide
RSSQSIVHSNGNTYLE

SEQ ID NO: 18 - protein, 16 aa, Artificial Sequence
  Description of Artificial Sequence Synthetic Peptide
RSSKSLLHSNGITYFF

SEQ ID NO: 19 - protein, 7 aa, Artificial Sequence
  Description of Artificial Sequence Synthetic Peptide
KVSNRFS

SEQ ID NO: 20 - protein, 7 aa, Artificial Sequence
  Description of Artificial Sequence Synthetic Peptide
QMSNLAS

SEQ ID NO: 21 - protein, 9 aa, Artificial Sequence
  Description of Artificial Sequence Synthetic Peptide
FQGSHVPLT

SEQ ID NO: 22 - protein, 9 aa, Artificial Sequence
  Description of Artificial Sequence Synthetic Peptide
AQNLELPPT

SEQ ID NO: 23 - protein, 5 aa, Artificial Sequence
  Description of Artificial Sequence Synthetic Peptide
NYWVN

SEQ ID NO: 24 - protein, 5 aa, Artificial Sequence
  Description of Artificial Sequence Synthetic Peptide
NYWIN

SEQ ID NO: 25 - protein, 5 aa, Artificial Sequence
  Description of Artificial Sequence Synthetic Peptide
NYWYN

SEQ ID NO: 26 - protein, 5 aa, Artificial Sequence
  Description of Artificial Sequence Synthetic Peptide
NYWWN

SEQ ID NO: 27 - protein, 5 aa, Artificial Sequence
  Description of Artificial Sequence Synthetic Peptide
DAWID

SEQ ID NO: 28 - protein, 5 aa, Artificial Sequence
  Description of Artificial Sequence Synthetic Peptide
DAWVD

SEQ ID NO: 29 - protein, 5 aa, Artificial Sequence
  Description of Artificial Sequence Synthetic Peptide
DAWYD

SEQ ID NO: 30 - protein, 5 aa, Artificial Sequence
  Description of Artificial Sequence Synthetic Peptide
DAWWD

SEQ ID NO: 31 - protein, 19 aa, Artificial Sequence
  Description of Artificial Sequence Synthetic Peptide
EIRSKANNYATYYAESVKG

SEQ ID NO: 32 - protein, 19 aa, Artificial Sequence
  Description of Artificial Sequence Synthetic Peptide
EIRLKSNKYTTHYAESVKG

SEQ ID NO: 33 - protein, 19 aa, Artificial Sequence
  Description of Artificial Sequence Synthetic Peptide
EIRLKSNSYTTHYAESVKG

SEQ ID NO: 34 - protein, 16 aa, Artificial Sequence
  Description of Artificial Sequence Synthetic Peptide
RPSQSIVHSNGNTYLE

SEQ ID NO: 35 - protein, 16 aa, Artificial Sequence
  Description of Artificial Sequence Synthetic Peptide
RSSQSIVHSNGNTYFE

SEQ ID NO: 36 - protein, 16 aa, Artificial Sequence
  Description of Artificial Sequence Synthetic Peptide
RPSQSIVHSNGNTYFE

SEQ ID NO: 37 - protein, 16 aa, Artificial Sequence
  Description of Artificial Sequence Synthetic Peptide
RPSKSLLHSNGITYFF

SEQ ID NO: 38 - protein, 16 aa, Artificial Sequence
  Description of Artificial Sequence Synthetic Peptide
RSSKSLLHSNGITYLF

SEQ ID NO: 39 - protein, 16 aa, Artificial Sequence
  Description of Artificial Sequence Synthetic Peptide
RPSKSLLHSNGITYLF

SEQ ID NO: 40 - protein, 9 aa, Artificial Sequence
  Description of Artificial Sequence Synthetic Peptide
FQGSHPPLT

SEQ ID NO: 41 - protein, 9 aa, Artificial Sequence
  Description of Artificial Sequence Synthetic Peptide
AQNLEPPPT

SEQ ID NO: 42 - protein, 5 aa, Artificial Sequence
  Description of Artificial Sequence Synthetic peptide
NYWLG

SEQ ID NO: 43 - protein, 17 aa, Artificial Sequence
  Description of Artificial Sequence Synthetic peptide
DIYPGGGYTNYNEKFKG

SEQ ID NO: 44 - protein, 17 aa, Artificial Sequence
  Description of Artificial Sequence Synthetic peptide
DIYPGGSYTNYNEKFKG

SEQ ID NO: 45 - protein, 10 aa, Artificial Sequence
  Description of Artificial Sequence Synthetic peptide
YDAAGPWFAY

SEQ ID NO: 46 - protein, 10 aa, Artificial Sequence
  Description of Artificial Sequence Synthetic peptide
YDAAGPGFAY

SEQ ID NO: 47 - protein, 8 aa, Artificial Sequence
  Description of Artificial Sequence Synthetic peptide
YDNHYFDY

SEQ ID NO: 48 - protein, 16 aa, Artificial Sequence
  Description of Artificial Sequence Synthetic peptide
RSSQSIVHSNGNTYLE

SEQ ID NO: 49 - protein, 16 aa, Artificial Sequence
  Description of Artificial Sequence Synthetic peptide
RSSQSLLHSNGNTYLH

SEQ ID NO: 50 - protein, 16 aa, Artificial Sequence
  Description of Artificial Sequence Synthetic peptide
KSSQSLLHSDGKTYLY

SEQ ID NO: 51 - protein, 7 aa, Artificial Sequence
  Description of Artificial Sequence Synthetic peptide
KVSNRFS

SEQ ID NO: 52 - protein, 7 aa, Artificial Sequence
  Description of Artificial Sequence Synthetic peptide
EVSSRFS

SEQ ID NO: 53 - protein, 9 aa, Artificial Sequence
  Description of Artificial Sequence Synthetic peptide
FQGSHVPYT

SEQ ID NO: 54 - protein, 9 aa, Artificial Sequence
  Description of Artificial Sequence Synthetic peptide
SQSTHVPYT

SEQ ID NO: 55 - protein, 5 aa, Artificial Sequence
  Description of Artificial Sequence Synthetic peptide
NYWIG

SEQ ID NO: 56 - protein, 5 aa, Artificial Sequence
  Description of Artificial Sequence Synthetic peptide
NYWMG

SEQ ID NO: 57 - protein, 5 aa, Artificial Sequence
  Description of Artificial Sequence Synthetic peptide
NYWWG

SEQ ID NO: 58 - protein, 5 aa, Artificial Sequence
  Description of Artificial Sequence Synthetic peptide
NYWVG

SEQ ID NO: 59 - protein, 17 aa, Artificial Sequence
  Description of Artificial Sequence Synthetic peptide
DIYPGGDYTNYNEKFKG

SEQ ID NO: 60 - protein, 17 aa, Artificial Sequence
  Description of Artificial Sequence Synthetic peptide
DIYPGGNYTNYNEKFKG

SEQ ID NO: 61 - protein, 17 aa, Artificial Sequence
  Description of Artificial Sequence Synthetic peptide
DIYTGGGYTNYNEKFKG

SEQ ID NO: 62 - protein, 17 aa, Artificial Sequence
  Description of Artificial Sequence Synthetic peptide
DIYTGGDYTNYNEKFKG

SEQ ID NO: 63 - protein, 17 aa, Artificial Sequence
  Description of Artificial Sequence Synthetic peptide
DIYTGGNYTNYNEKFKG

SEQ ID NO: 64 - protein, 17 aa, Artificial Sequence
  Description of Artificial Sequence Synthetic peptide
DIYTGGSYTNYNEKFKG

SEQ ID NO: 65 - protein, 17 aa, Artificial Sequence
  Description of Artificial Sequence Synthetic peptide
DIYAGGGYTNYNEKFKG

SEQ ID NO: 66 - protein, 17 aa, Artificial Sequence
  Description of Artificial Sequence Synthetic peptide
DIYAGGDYTNYNEKFKG

SEQ ID NO: 67 - protein, 17 aa, Artificial Sequence
  Description of Artificial Sequence Synthetic peptide
DIYAGGDYTNYNEKFKG

SEQ ID NO: 68 - protein, 17 aa, Artificial Sequence
  Description of Artificial Sequence Synthetic peptide
DIYAGGSYTNYNEKFKG

SEQ ID NO: 69 - protein, 16 aa, Artificial Sequence
  Description of Artificial Sequence Synthetic peptide
RPSQSIVHSNGNTYLE

SEQ ID NO: 70 - protein, 16 aa, Artificial Sequence
  Description of Artificial Sequence Synthetic peptide
RPSQSIVHSNGNTYLE

SEQ ID NO: 71 - protein, 16 aa, Artificial Sequence
  Description of Artificial Sequence Synthetic peptide
RSSQSIVHSNGNTYFE

SEQ ID NO: 72 - protein, 16 aa, Artificial Sequence
  Description of Artificial Sequence Synthetic peptide
RPSQSLVHSNGNTYLE

SEQ ID NO: 73 - protein, 16 aa, Artificial Sequence
  Description of Artificial Sequence Synthetic peptide
RPSQSIVHSNGNTYFE

SEQ ID NO: 74 - protein, 16 aa, Artificial Sequence
  Description of Artificial Sequence Synthetic peptide
RSSQSLVHSNGNTYFE

SEQ ID NO: 75 - protein, 16 aa, Artificial Sequence
  Description of Artificial Sequence Synthetic peptide
RPSQSLLHSNGNTYLH

SEQ ID NO: 76 - protein, 16 aa, Artificial Sequence
  Description of Artificial Sequence Synthetic peptide
RSSQSILHSNGNTYLH

SEQ ID NO: 77 - protein, 16 aa, Artificial Sequence
  Description of Artificial Sequence Synthetic peptide
RSSQSLLHSNGNTYFH

SEQ ID NO: 78 - protein, 16 aa, Artificial Sequence
  Description of Artificial Sequence Synthetic peptide
RPSQSILHSNGNTYLH

SEQ ID NO: 79 - protein, 16 aa, Artificial Sequence
  Description of Artificial Sequence Synthetic peptide
RPSQSLLHSNGNTYFH

SEQ ID NO: 80 - protein, 16 aa, Artificial Sequence
  Description of Artificial Sequence Synthetic peptide
RSSQSILHSNGNTYFH

SEQ ID NO: 81 - protein, 16 aa, Artificial Sequence
  Description of Artificial Sequence Synthetic peptide
KPSQSLLHSDGKTYLY

SEQ ID NO: 82 - protein, 16 aa, Artificial Sequence
  Description of Artificial Sequence Synthetic peptide
KSSQSILHSDGKTYLY

SEQ ID NO: 83 - protein, 16 aa, Artificial Sequence
  Description of Artificial Sequence Synthetic peptide
KSSQSLLHSDGKTYFY

SEQ ID NO: 84 - protein, 16 aa, Artificial Sequence
  Description of Artificial Sequence Synthetic peptide
KPSQSILHSDGKTYLY

SEQ ID NO: 85 - protein, 16 aa, Artificial Sequence
  Description of Artificial Sequence Synthetic peptide
KPSQSILHSDGKTYLY

SEQ ID NO: 86 - protein, 16 aa, Artificial Sequence
  Description of Artificial Sequence Synthetic peptide
KSSQSILHSDGKTYFY

(19) United States
(12) Patent Application Publication
(10) Pub. No.: US 2018/0028680 A1
(43) Pub. Date: Feb. 1, 2018

(54) GLYCOOPTIMIZED ANTIBODY DRUG CONJUGATES

(71) Applicant: GLYCOTOPE GMBH, Berlin (DE)

(72) Inventors: Felix HART, Berlin (DE); Antje DANIELCZYK, Berlin (DE); Steffen GOLETZ, Berlin (DE)

(21) Appl. No.: 15/552,357

(22) PCT Filed: Feb. 22, 2016
(86) PCT No.: PCT/EP2016/053632

§ 371 Date: Aug. 21, 2017

(30) Foreign Application Priority Data
Feb. 23, 2015 (LU) 92659

(51) Int. Cl. A61K 47/68; C07K 16/30; C07K 16/32

(52) U.S. Cl. CPC A61K 47/6851; C07K 16/32; C07K 16/30; C07K 2317/41; C07K 2317/732; C07K 2317/565; C07K 2317/56; C07K 2317/77; C07K 2317/76

(57) ABSTRACT

The present invention relates to antibody-drug conjugates (ADCs) and a method for improving the safety profile of the same, comprising linking an antibody molecule to a drug in order to obtain said ADC, wherein said antibody molecule is obtainable from a host cell selected to produce an antibody molecule composition having specific glycosylation

### BACKGROUND

Antibody therapeutics have revolutionized the treatment of cancer over the past two decades. Antibodies that specifically bind tumor surface antigens can be effective therapeutics; however, many unmodified antibodies lack therapeutic activity. These antibodies can instead be applied successfully as guided missiles to deliver potent cytotoxic drugs in the form of antibody drug conjugates (ADCs). These ADCs are a promising therapeutic modality delivering medicine to patients suffering from a variety of malignancies. ADCs consist of three different components (antibody, linker, and drug/cytotoxic payload) that are responsible for the targeted delivery of payload specifically to the cancer cells. Delivery of cytotoxic agents to the tumor, e.g. via tumor-specific overexpressed cell surface antigens, improves the efficacy and selectivity of the payload. The targeted delivery of the payload also minimizes the normal tissue exposure of the payload, resulting in decreased toxicity and improved therapeutic index of the payload. Frequently used payloads fall into two categories: microtubule inhibitors and DNA-damaging agents.

Once taken up into cognate antigen-expressing tumor cells, the drug is released through mechanisms that depend on which type of linker is used from the antibody-drug conjugate. The drug can then kill tumor cells through its established cytotoxic mechanisms. Alternatively, antibodies can be fused directly to cytokines; these antibody-drug conjugates can act extracellularly by recruiting cytotoxic immune cells to the tumor site, thereby indirectly killing tumor cells. Some antibody-drug conjugates have been approved for clinical use in a variety of solid and hematological tumors, and many more are in clinical trials. In general, antibody-drug conjugates may provide a way to repurpose tumor-specific antibodies, which on their own did not have therapeutic activity, or chemotherapeutic drugs, which when injected systemically, are too toxic for healthy tissues. Success of ADCs is dependent on four factors—target antigen, antibody, linker, and payload.

The field has made great progress in these areas, marked by the recent approval by the US Food and Drug Administration of two ADCs, brentuximab vedotin (Adcetris®) and ado-trastuzumab emtansine (Kadcyla®). However, the therapeutic window for many ADCs that are currently in pre-clinical or clinical development remains narrow and further improvements may be required to enhance the therapeutic potential of these ADCs. In fact, it may thus be advantageous if ADCs, apart from acting as target-seeking molecular missiles with lethal payload, have additional functions such as mediating antibody-dependent cell cytotoxicity (ADCC). ADCC is mediated by the Fc portion of an antibody.

ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response can act to limit and contain infection. Classical ADCC is mediated by natural killer (NK) cells; macrophages, neutrophils and eosinophils can also mediate ADCC. The typical ADCC involves activation of NK cells by antibodies. An NK cell expresses CD16 which is an Fc receptor. This receptor recognizes, and binds to, the Fc portion of an antibody, such as IgG, which has bound to the surface of a pathogen-infected target cell. The most common Fc receptor on the surface of an NK cell is called CD16 or FcyRlll. Once the Fc receptor binds to the Fc region of IgG, the Natural Killer cell releases cytokines such as IFN-gamma.

However, ADCC does not occur under all circumstances. For example, the density of the antigen on the surface of a target cell may be too low such that too less antibodies bind and thus immune effector cells may not take action. Also, the amount of antibody that is or can be administered to a patient may not be sufficient to occupy target cells such that again immune effector cells take action. Yet, though the latter issue may theoretically be overcome by increasing the dose and thus amount of an ADC, there are limits. For example, ADCs may not be administered in high doses, because there is an inherent risk that ADC may release their highly toxic payload before it reaches its target and is internalized. The payload may thus cause undesired or even deleterious side effects. Similarly, patients may simply be overdosed when administering high doses of an ADC with the aim of densely occupying target cells in order to mediate ADCC. Hence, it would be highly desirable to have available ADCs that are administered at doses which are safe for patient, but which still have the capacity to mediate ADCC to an extent which, in addition to the payload of the ADC, contributes to the killing of a target cell, i.e. an ideally bi-functional mode of action ADC such that both modes of action act together or one mode of action may complement the other, if for certain reasons, one mode would fail.

Accordingly, the technical problem underlying the present application is to comply with the unmet needs existing in the prior art. The technical problem is solved by the provision of the methods, uses and means described herein below, reflected in the claims, exemplified in the Examples and illustrated in the Figures.

The present invention relates to method for improving the safety profile and/or efficacy of an antibody-drug-conjugate (ADC), comprising linking an antibody molecule to a drug in order to obtain said ADC, said antibody molecule being obtainable from a host cell selected to produce an antibody molecule composition having at least one of the following characteristics:
it comprises no detectable NeuGc; and/or
it has a galactosylation degree on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same protein molecule isolated from ATCC No. CRL-9096 (CHOdhfr-) when expressed therein; and/or
it has an amount of GO structures on the total carbohydrate structures or on the carbohydrate structures at one particular glycosylation site of the protein molecule of said protein molecules in said protein molecule composition which is at least 5% lower compared to the same amount of protein molecules in at least one protein molecule composition of the same protein molecule isolated from ATCC No. CRL-9096 (CHOdhfr-) when expressed therein; and/or
it comprises no detectable terminal Galalphal-3Gal; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein; and/or
it comprises at least one carbohydrate structure containing bisecting GlcNAc; and/or
it has a sialylation pattern which is altered compared to the sialylation pattern of at least one antibody molecule composition of the same antibody molecule isolated from ATCC No. CRL-9096 (CHOdhfr-) when expressed therein, wherein improvement of said safety profile and/or efficacy allows a reduction of the dose of said ADC to be administered to a patient in comparison to an ADC, the antibody part of which was preferably not obtained from a host cell having at least one of the glycosylation characteristics as defined above.

Put differently, linking an antibody molecule as described herein, i.e. produced by a host having the characteristics as described herein, to a drug in order to obtain an ADC thereby allows an improvement of the safety profile and/or efficacy in that the dose of said ADC to be administered to a patient can be reduced.

However, it is also envisaged that the antibody part of said ADC was obtained from a host cell having at least one of the glycosylation characteristics as defined above. In fact, as regards the latter embodiment, it was observed by the present inventors that ADCs, the antibody of which was obtained from a host cell having at least one of the glycosylation characteristics as defined above can be administered at lower doses, while their efficacy is maintained or even improved.

In some embodiments, said sialylation pattern is characterized by at least one of the following characteristics:
it comprises alpha2-6 linked NeuNAc; and/or
it has an increased sialylation degree with an amount of NeuNAc on the total carbohydrate structures or on the carbohydrate structures at one particular glycosylation site of the antibody molecule of the antibody molecules in said antibody molecule composition which is at least 15% higher compared to the same amount of antibody molecules in at least one antibody molecule composition of the same antibody molecule isolated from ATCC No. CRL-9096 (CHOdhfr-) when expressed therein, and/or
it comprises at least 20% more charged N-glycosidically linked carbohydrate chains of the total carbohydrate units or of at least one particular carbohydrate chain at a particular glycosylation site of the antibody molecule of the antibody molecules in said antibody molecule composition compared to the same amount of antibody molecules in at least one antibody molecule composition of the same antibody molecule isolated from ATCC No. CRL-9096 (CHOdhfr-) when expressed therein.

In some embodiments, said host cell is selected to produce an antibody comprising
at least 10% carbohydrate structures of the total carbohydrate units or of at least one particular carbohydrate chain at a particular glycosylation site of the antibody molecule of the antibody molecules in said antibody molecule composition, lacking fucose; and/or
at least 2% carbohydrate structures of the total carbohydrate units or of at least one particular carbohydrate chain at a particular glycosylation site of the antibody molecule of the antibody molecules in said antibody molecule composition which contains bisecting GlcNAc; and/or
more than 35% G2 structures on the total carbohydrate structures or on the carbohydrate structures at one particular glycosylation site of the antibody molecule in said antibody molecule composition; and/or
it comprises less than 22% G0 structures on the total carbohydrate structures or on the carbohydrate structures at one particular glycosylation site of the antibody molecule in said antibody molecule composition.

In some embodiments, said host cell is selected to produce an antibody having the following characteristic glycosylation combinations:
(a)
   it comprises no detectable NeuGc
   it comprises no detectable Galalpha1-3Gal
   it comprises a galactosylation pattern as defined herein
   it has a fucose content as defined in herein
   it comprises bisecGlcNAc
   it comprises an increased amount of sialic acid compared to a antibody composition of the same antibody molecule when expressed in the cell line ATCC No. CRL-9096 (CHOdhfr-) or compared to a sialylation deficient cell line such as DSM ACC2606 (NM-F9) and DSM ACC2605 (NM-D4); or
(b)
   it comprises no detectable NeuGc
   it comprises no detectable Galalpha1-3Gal
   it comprises a galactosylation pattern as defined in herein
   it has a fucose content as defined in herein
   it comprises bisecGlcNAc
   it comprises 2-6 NeuNAc.

In some embodiments, said antibody
comprises no detectable NeuGc;
comprises a2,6-linked NeuNAc; and
has an increased sialylation degree with an amount of NeuNAc on the total carbohydrate structures or on the carbohydrate structures at one particular glycosylation site of the antibody molecule of said antibody molecules in said antibody molecule composition which is at least 15% higher compared to the same amount of antibody molecules in at least one antibody molecule composition of the same antibody molecule isolated from ATCC No. CRL-9096 (CHOdhfr-) when expressed therein.

In some embodiments the antibody comprises at least 2%, preferably at least 5%, more preferably at least 10% and most preferably at least 15% carbohydrate structures of the total carbohydrate units or of at least one particular carbohydrate chain at a particular glycosylation site of an antibody molecule of the antibody molecules in said antibody molecule composition which contains bisecting GlcNAc.

In some embodiments, the antibody has an increased sialylation degree with an amount of NeuNAc on the total carbohydrate structures or on the carbohydrate structures at one particular glycosylation site of the antibody molecule of said antibody molecules in said antibody molecule composition which is at least 20%, preferably at least 30% higher compared to the same amount of antibody molecules in at least one antibody molecule composition of the same antibody molecule isolated from ATCC No. CRL-9096 (CHOdhfr-) when expressed therein.

In some embodiments, the antibody comprises at least 50%, preferably at least 60% and more preferably at least 70% carbohydrate structures of the total carbohydrate units or of at least one particular carbohydrate chain at a particular glycosylation site of an antibody molecule of the antibody molecules in said antibody molecule composition, lacking fucose.

In some embodiments, the antibody molecule composition comprises no detectable terminal Gala/pha1-3Gal.

In some embodiments, the antibody molecule has at least one of the following characteristics
it has a galactosylation degree of galactose, which is linked to GlcNAc, on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same antibody molecule isolated from ATCC No. CRL-9096 (CHOdhfr-) when expressed therein; and/or
it has an amount of GO structures on the total carbohydrate structures or on the carbohydrate structures at one particular glycosylation site of the antibody molecule of said antibody molecules in said antibody molecule composition which is at least 5% lower compared to the same amount of antibody molecules in at least one antibody molecule composition of the same antibody molecule isolated from ATCC No. CRL-9096 (CHOdhfr-) when expressed therein; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein.
it comprises at least 20% more charged N-glycosidically linked carbohydrate chains of the total carbohydrate units or of at least one particular carbohydrate chain at a particular glycosylation site of the antibody molecule of the antibody molecules in said antibody molecule composition compared to the same amount of antibody molecules in at least one antibody molecule composition of the same antibody molecule isolated from ATCC No. CRL-9096 (CHOdhfr-) when expressed therein, and/or
it comprises more than 35% G2 structures on the total carbohydrate structures or on the carbohydrate structures at one particular glycosylation site of an antibody molecule of the antibody molecules in said antibody composition; and/or
it comprises less than 22% GO structures on the total carbohydrate structures or on the carbohydrate structures at one particular glycosylation site of an antibody molecule of the antibody molecules in said antibody composition; and/or
it has an increased activity and/or increased yield compared to at least one antibody molecule composition of the same antibody molecule when expressed in the cell line ATCC No. CRL-9096 (CHOdhfr-); and/or
it has an improved homogeneity compared to at least one antibody molecule composition of the same antibody molecule when expressed in the cell line ATCC No. CRL-9096 (CHOdhfr-); and/or
it has an increased activity which is at least 10% higher than the activity of at least one antibody molecule composition from the same antibody molecule when expressed in the cell line ATCC No. CRL-9096 (CHOdhfr-); and/or
it has an increased Fc-mediated cellular cytotoxicity which is at least 2 to 5, such as 2, 3, 4, or 5 times higher than the Fc-mediated cellular cytotoxicity of at least one antibody molecule composition from the same antibody molecule when expressed in the cell line ATCC No. CRL-9096 (CHOdhfr-); and/or
it has an increased antigen mediated or Fc-mediated binding which is at least 50% higher than the binding of at least one antibody molecule composition from the same antibody molecule when expressed in the cell line ATCC No. CRL-9096 (CHOdhfr-); and/or - it has ADCC and/or CDC activity.

In some embodiments, the host cell is selected from the group consisting of NM-F9 [DSM ACC2606], NM-D4 [DSM ACC2605], GT-2X [DSM ACC2858], NM-H9, NM-E-2F9, NM-C-2F5, NM-H9D8 [DSM ACC2806], NM-H9D8-E6 [DSM ACC2807], NM-H9D8-E6Q12 [DSM ACC2856], GT-5s [DSM ACC 3078] or a cell or cell line derived therefrom.

The present invention further relates to an ADC obtainable by the method as described herein for use in a method of treatment of cancer, said method comprising administering said ADC at doses which are lower than doses for an ADC, the antibody part of which was preferably not obtained from a host cell having at least one of the glycosylation characteristics as defined above. Preferably, at said lower doses the Fc receptor (FcR) binding of said ADC is at least equal to or even improved in comparison an ADC, the antibody part of which was preferably not obtained from a host cell having at least one of the glycosylation characteristics as defined above. However, it is also envisaged that the antibody part of said ADC was obtained from a host cell having at least one of the glycosylation characteristics as defined above. In fact, as regards the latter embodiment, it was observed by the present inventors that ADCs, the antibody part of which was obtained from a host cell having at least one of the glycosylation characteristics as defined above can be administered at lower doses, while their efficacy is maintained or even improved. It is generally preferred in the context of the present invention and particularly preferred for an ADC obtainable by the method as described herein for use in a method of treatment of cancer that the antibody part of said ADC is not an antibody directed to HER-2 or HER2/neu, BCMA, tenascin A2 and the A2 domain of tenascin A2 (TNC A2). It is generally preferred in the context of the present invention and particularly preferred for an ADC obtainable by the method as described herein for use in a method of treatment of cancer that the antibody part of said ADC is not an antibody directed to HER-2 or HER2/neu, BCMA, tenascin A2 or the A2 domain of tenascin A2 (TNC A2).

In some embodiments, the ADC has enhanced Fc receptor (FcR) binding resulting in enhanced immunological effector functions of said ADC, said enhanced FcR binding is enhanced to the extent such that said ADC mediates enhanced immunological effector functions at doses at which essentially no immunological effector functions are mediated in comparison to said ADC without enhanced FcR binding. In particular, said enhanced FcR binding is envisaged to be enhanced Fc gamma receptor binding, particularly Fc gamma receptor III binding, and/or mediates enhanced antibody dependent cell cytotoxicity (ADCC).

In some embodiments, the antibody of said ADC is directed against a molecule on the surface of a cancer cell, said cancer preferably being characterized by a solid tumor such as breast cancer tumor.

In some embodiments, the antibody of the ADC comprises
(i) a heavy chain variable region comprising a CDR1 having the amino acid sequence of SEQ ID NO: 1 , a CDR2 having the amino acid sequence of SEQ ID NO: 2, and a CDR3 having the amino acid sequence of SEQ ID NO: 3; and
(ii) a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6.

In some embodiments, the antibody of the ADC comprises
(i) a heavy chain variable region comprising the amino acid sequence of SEQ ID NO: 7 or an amino acid sequence which is at least 80% identical thereto; and
(ii) a light chain variable region comprising the amino acid sequence of SEQ ID NO: 8 or an amino acid sequence which is at least 80% identical thereto.

In some embodiments, the antibody of the ADC comprises
(i) a heavy chain comprising the amino acid sequence of SEQ ID NO: 9 or an amino acid sequence which is at least 80% identical thereto; and
(ii) a light chain variable region comprising the amino acid sequence of SEQ ID NO: 10 or an amino acid sequence which is at least 80% identical thereto.

The present invention also provides for the ex vivo use of a host cell as described herein for improving the safety profile and/or efficacy of an antibody-drug-conjugate (ADC). Thus, by using a host cell having the characteristics as described for the (ex vivo) production of the antibody part of said ADC, the safety profile and/or efficacy of an antibody-drug-conjugate can be improved.

In some embodiments, an improvement is characterized by a reduction of the dose of an ADC to be administered to a patient in comparison to an ADC, the antibody part of which was preferably not obtained from a host cell having at least one of the glycosylation characteristics as described herein. However, it is also envisaged that the antibody part of said ADC was obtained from a host cell having at least one of the glycosylation characteristics as defined above. In fact, as regards the latter embodiment, it was observed by the present inventors that ADCs, the antibody part of which was obtained from a host cell having at least one of the glycosylation characteristics as defined above can be administered at lower doses, while their efficacy is maintained or even improved

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

The term “less than” or “greater than” includes the concrete number. For example, less than 20 means less than or equal to. Similarly, more than or greater than means more than or equal to, or greater than or equal to, respectively.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### DESCRIPTION OF THE FIGURES

FIG. 1: Comparison of glycooptimized antibody against Her2 TrasGEX with Herceptin in antigen binding on tumor cells and Fv-mediated anti-tumor activities as inhibition of proliferation, VEGF inhibition, receptor down-modulation, and apoptosis induction.

FIG. 2: ADCC assays on SK-BR-3 and on MCF-7 cells with TrasGEX and Herceptin using primary human PBMC of donors with different FcgRIIIA allotypes (V/V and F/F).

FIG. 3: Determination of antibody concentrations needed for same lysis (50% of maximal lysis of the improved antibody)

FIG. 4: Internalization of a glycooptimized antibody against Her2 into acidic compartments of cancer cells

FIG. 5: Sequences of the present invention

### DETAILED DESCRIPTION

### Advantages

The present inventors found that they can improve the safety potential and/or efficacy of ADCs and, thereby, allowing a reduction of the dose of ADCs by equipping ADCs with high ADCC such that ADCs have, even at low doses, the potential to deliver their payload, but in particular mediate ADCC by their enhanced Fc receptor binding. At such low doses, ADCC is usually not mediated by antibodies. Hence, ADCs of the present invention have at even lower doses at least the same or an even improved efficacy in comparison to ADCs, the antibody part of which is preferably not produced in accordance with the teaching of the present invention. Accordingly, in contrast to prior art ADCs, ADCs of the present invention can be administered at reduced doses, while their efficacy is maintained or even improved.

Accordingly, it is an advantage of the ADCs of the present invention that they can be administering at doses which are lower than doses for an ADC which was not obtained from a host cell having at least one of the glycosylation characteristics of a host cell as described herein. Without being bound by theory, it is assumed that ADCs of the present invention can be administered in doses that are, for example, below the doses of the so far approved ADCs—T-DM1 (3.6 mg/kg; q3w) an Adcetris (1.8 mg/kg; q3w).

The improvement achieved by the present invention is insofar pioneering as it will likely allow administration of ADCs at low doses, while keeping their functionality, in particular their property of having an enhanced Fc receptor binding, particularly Fc gamma receptor III binding and/or mediating ADCC. In particular, this improvement is achieved by applying an antibody (as part of the ADC) having at least one of the characteristics as described herein elsewhere and/or that is producible by a host cell having at least one of the following characteristics
it comprises no detectable NeuGc; and/or
it has a galactosylation degree on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same antibody molecule isolated from ATCC No. CRL-9096 (CHOdhfr-) when expressed therein; and/or
it has an amount of GO structures on the total carbohydrate structures or on the carbohydrate structures at one particular glycosylation site of the antibody molecule of said antibody molecules in said antibody molecule composition which is at least 5% lower compared to the same amount of antibody molecules in at least one antibody molecule composition of the same antibody molecule isolated from ATCC No. CRL-9096 (CHOdhfr-) when expressed therein; and/or
it comprises no detectable terminal Galalpha1-3Gal; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein; and/or
it comprises at least one carbohydrate structure containing bisecting GlcNAc; and/or
it has a sialylation pattern which is altered compared to the sialylation pattern of at least one antibody molecule composition of the same antibody molecule isolated from ATCC No. CRL-9096 (CHOdhfr-) when expressed therein

### Antibody

In a first aspect, the present invention relates to a method for improving the safety profile and/or efficacy of an antibody-drug-conjugate (ADC), comprising linking an antibody to a drug in order to obtain said ADC, said antibody being obtainable from a host cell selected to produce an antibody having the characteristics as described elsewhere herein.

The term “antibody drug conjugate”, abbreviated “ADC” (said term can be interchangeable used with the term “immunoconjugate”) as used herein refers to the linkage of an antibody thereof with a drug or agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the ADC as is known in the art and described herein. An ADC as used in the context of the present invention comprises an antibody and a drug that is linked to said antibody.

The term “antibody” is used herein interchangeably with the term “antibody molecule” and in particular refers to a protein comprising at least two heavy chains and two light chains connected by disulfide bonds. An antibody when referred to herein is preferably a part or component of an antibody drug conjugate (ADC). Each heavy chain is comprised of a heavy chain variable region (VH) and a heavy chain constant region (CH). Each light chain is comprised of a light chain variable region (VL) and a light chain constant region (CL). The heavy chain-constant region comprises three or -in the case of antibodies of the IgM- or IgE-type-four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen The heavy chain constant regions may be of any type such as γ-, δ-, α-, μ- or ε-type heavy chains. The heavy chain of the antibody may in particular be a γ-chain. Furthermore, the light chain constant region may also be of any type such as κ- or λ-type light chains. The light chain of the antibody may in particular be a κ-chain. The constant regions of the antibodies typically mediate the binding of the immunoglobulin to host tissues or factors, including various cells of the immune system (e.g., effector cells) and the first component (C1 q) of the classical complement system. The variable regions of the heavy and light chains typically contain a binding domain that interacts with an antigen. The antibody can be e.g. a humanized, human or chimeric antibody. The antibody being part of an ADC according to the invention is preferably capable of inducing ADCC.

The term “antibody” according to the invention includes antibodies such as heavy chain antibodies, i.e. antibodies only composed of one or more, in particular two heavy chains, nanobodies, i.e. antibodies only composed of a single monomeric variable domain, or antibody fragments or derivatives. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. Examples of fragments or derivatives of an antibody include (i) Fab fragments, monovalent fragments consisting of the variable region and the first constant domain of each the heavy and the light chain; (ii) F(ab)2 fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)2 fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. These antibody fragments and derivatives are obtained using conventional techniques known to those with skill in the art.

The “Fab part” of an antibody in particular refers to a part of the antibody comprising the heavy and light chain variable regions (VH and VL) and the first heavy and light chain constant regions (CH1 and CL). In cases where the antibody does not comprise all of these regions, then the term “Fab part” only refers to those of the regions VH, VL, CH1 and CL which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the VH region of the antibody.

The “Fc part” of an antibody in particular refers to a part of the antibody comprising the heavy chain constant regions 2, 3 and —where applicable—4 (CH2, CH3 and CH4). In cases where the antibody does not comprise all of these regions, then the term “Fc part” only refers to those of the regions CH2, CH3 and CH4 which are present in the antibody. Preferably, the Fc part comprises at least the CH2 region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises a Fc receptor binding site or a Fc receptor binding ability. e or a Fc receptor binding ability. Furthermore, preferably it is capable of inducing ADCC. Preferably, the Fc part comprises at least the CH2 region of the antibody.

In particular, the antibody may be of any isotype such as IgA, IgD, IgE, IgG or IgM, including any subclass such as IgG1 , IgG2, IgG3, IgG4, IgA1 or IgA2. The antibody may in particular be an IgG1 - or IgG2-antibody, more preferably an IgG1 -antibody. For indicating the amino acid positions of the heavy chain and light chain, in particular the variable regions thereof, the Kabat numbering system is used herein (Kabat, E.A. et al. (1991) Sequences of Proteins of Immunological Interest, 5th edition, NIH Publication No. 91 -3242). According to said system, the heavy chain variable region comprises amino acid positions from position 0 to position 1 13 including position 35A, 35B, 52A to 52C, 82A to 82C and 100A to 100K. The CDRs of the heavy chain variable region are located, according to the Kabat numbering, at positions 31 to 35B (CDR1), 50 to 65 (CDR2) and 95 to 102 (CDR3). The remaining amino acid positions form the framework regions FR1 to FR4. The light chain variable region comprises positions 0 to 109 including positions 27A to 27F, 95A to 95F and 106A. The CDRs are located at positions 24 to 34 (CDR1), 50 to 56 (CDR2) and 89 to 97 (CDR3). Depending on the initial formation of the specific gene of an antibody, not all of these positions have to be present in a given heavy chain variable region or light chain variable region. In case an amino acid position in a heavy chain or light chain variable region is mentioned herein, unless otherwise indicated it is referred to the position according to the Kabat numbering.

The term “antibody” includes chimeric and humanized antibodies. According to the present invention, the term “chimeric antibody” in particular refers to an antibody wherein the constant regions are derived from a human antibody or a human antibody consensus sequence, and wherein at least one and preferably both variable regions are derived from a non-human antibody, e.g. from a rodent antibody such as a mouse antibody.

The term “humanized antibody” in particular refers to an antibody wherein at least one CDR is derived from a non-human antibody, and wherein the constant regions, if present, and at least one framework region of a variable region are derived from a human antibody or a human antibody consensus sequence. Methods for constructing humanized antibodies are known to the one skilled in the art and include CDR grafting, resurfacing, superhumanization, and human string content optimization. Overviews of humanization processes can be found, for example, in Almagro, J. C. and Fransson, J. (2008) Frontiers in Bioscience 13, 1619-1633 and in the entire volume 36 of the Journal Methods (2005).

Furthermore, the antibody according to the present invention may have been subjected to framework or Fc engineering. Such engineered antibodies include those in which modifications have been made to framework residues within V_{H} and/or V_{L}, e.g. to improve the properties of the antibody. Typically such framework modifications are made to decrease the immunogenicity of the antibody.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95% or at least 97%. For example, if a framework region of a humanized antibody is derived from or corresponds to a variable region of a particular human antibody, then the amino acid of the framework region of the humanized antibody shares a homology or identity over its entire length with the corresponding framework region of the human antibody of at least 75%, more preferably at least 80%, at least 85%, at least 90%, at least 93%, at least 95% or at least 97%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined.

A target amino acid sequence is “derived” from a reference amino acid sequence if the target amino acid sequence shares a homology or identity over its entire length with a corresponding part of the reference amino acid sequence of at least 60%, preferably at least 70%, at least 75%, more preferably at least 80%, at least 85%, at least 90%, at least 93% , at least 95% or at least 97%. For example, if a framework region of a humanized antibody is derived from a variable region of a particular human antibody, then the amino acid of the framework region of the humanized antibody shares a homology or identity over its entire length with the corresponding framework region of the human antibody of at least 60%, preferably at least 70%, at least 75%, more preferably at least 80%, at least 85%, at least 90%, at least 93%, at least 95% or at least 97%. The “corresponding part” or “corresponding framework region” means that, for example, framework region 1 of a heavy chain variable region (FRH1) of a target antibody corresponds to framework region 1 of the heavy chain variable region of the reference antibody. The same is true, for example, for FRH2, FRH3, FRH4, FRL1, FRL2, FRL3 and FRL4. In particular embodiments, a target amino acid sequence which is “derived” from a reference amino acid sequence is 100% homologous, or in particular 100% identical, over its entire length with a corresponding part of the reference amino acid sequence.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with a Kₐ of at least 10⁶ M″¹, preferably at least 10⁷ M″¹, more preferably at least 10⁸ M″¹. An antibody specific for a certain antigen in particular refers to an antibody which is capable of binding to said antigen with an affinity having a Kₐ of at least 10⁶ M″¹, preferably at least 10⁷ M″¹, more preferably at least 10⁸ M″¹. For example, the term “anti-EGFR antibody” refers to an antibody specifically binding EGFR and preferably is capable of binding to EGFR with an affinity having a Kₐ of at least 10⁶ M′¹, preferably at least 10⁷ M′¹, more preferably at least 10⁸ M″¹.

The term “antibody”, as used herein, refers in certain embodiments to a population of antibodies of the same kind. In particular, all antibodies of the population of the antibody exhibit the features used for defining the antibody. In certain embodiments, all antibodies in the population of the antibody have the same amino acid sequence. Reference to a specific kind of antibody, such as an anti-EGFR antibody or a reduced fucose anti-EGFR antibody, in particular refers to a population of this kind of antibody.

### Antibody Characteristics

The inventive method comprises the steps of linking an antibody molecule to a drug in order to obtain an antibody-drug-construct (ADC), said antibody molecule being obtainable or being producible from a host cell selected to produce an antibody molecule composition having specific characteristics as described herein. The host cell is selected to produce an antibody molecule composition having at least one of the following characteristics (“possible characteristics”). It is envisaged that the antibody molecules exhibit and retain at least one of said characteristics even after being linked to the drug in the ADC. The characteristics described in the following may be present individually or cumulatively in any combination.

### NeuGc

One possible characteristic of the antibody is that it comprises no detectable NeuGc. The term “NeuGc” as used herein refers to N-glycolylneuraminic acid. Most rodent cells such as CHO, BHK, NSO, Sp2/0 and YB2/0 express for example NeuGc as an alternative for N-acetylneuraminic acid (“NeuNAc”). However, rodent cells are not generally excluded from the inventive methods as long as they produce antibodies with no detectable NeuGc. Without wishing to be bound by theory, NeuGc glycosylation may have immunogenic properties in humans. Hence, it is desirable to avoid a respective glycosylation as far as possible. A respective glycosylation can, e.g., be avoided by using immortalized human blood cells and in particular by using a host cell of human myeloid leukaemia origin.

“No detectable NeuGc” does not necessarily mean that there is absolutely no NeuGc present. Conversely, also embodiments are encompassed, which have a rather low degree of NeuGc (e.g. 1 to 10%).

### Galactosylation

Another possible characteristic of the antibody is that it has an increased galactosylation degree, meaning that it has a galactosylation degree on the total carbohydrate structures or on the carbohydrate structures at one particular glycosylation site of the antibody molecule of the antibody molecules in the antibody molecule composition, that is increased compared to the same amount of antibody molecules in at least one antibody molecule composition of the same antibody molecule isolated from ATCC No. CRL-9096 (CHOdhfr-) when expressed therein.

As used herein, the term “glycosylation site” in particular refers to an amino acid sequence which can specifically be recognized and glycosylated by a natural glycosylation enzyme, in particular a glycosyltransferase, preferably a naturally occurring mammalian or human glycosyltransferase. In particular, the term “glycosylation site” refers to an N-glycosylation site, comprising an asparagine residue to which the carbohydrate is or can be bound. In particular, the glycosylation site is an N-glycosylation site which has the amino acid sequence Asn-Xaa-Ser/Thr/Cys, wherein Xaa is any amino acid residue. Preferably, Xaa is not Pro.

The galactose residues are found mainly beta 1-4 linked to the GlcNAc residues on the antennas of the complex type N-glycan of antibodies, but also beta-1 ,3 linkages have been found. However, they usually occur in triantennary structures. The influence of the degree of galactosylation on the activity is in particular regarding antibodies remarkable. It has been demonstrated that depletion of galactose leads to a reduced CDC activity. Hence, it may be preferred to have a high degree of galactosylation. Galactosylation may also play an important role for other proteins.

When referring to the total carbohydrate structure of a protein molecule, all glycosylations of the antibody molecule are considered. In case the carbohydrate structures at one particular glycosylation site of the protein molecule is analysed, the focus lies on a specific carbohydrate structure(s), such as e.g. the carbohydrate structure(s) attached to the Asn 297 of the Fc part of an antibody molecule. In case a respective specific structure is evaluated, the content/composition of this specific structure is determined. One could also refer to total carbohydrate units and particular carbohydrate chains for defining said characteristics (these are synonyms).

### G2 Structures

Another possible characteristic of the antibody is that has an increased amount of G2 structures, meaning that it has an amount of G2 structures on the total carbohydrate structures or on the carbohydrate structures at one particular glycosylation site of the antibody molecule of the antibody molecules in the antibody molecule composition which is at least 5% higher compared to the same amount of protein molecules in at least one antibody molecule composition of the same antibody molecule isolated from ATCC No. CRL-9096 (CHOdhfr-) when expressed therein.

In particular, in case an antibody is produced according to the methods of the present invention, a high amount of G2 structures is beneficial. A “G2 structure” defines a glycosylation pattern wherein galactose is found at both ends of the biantennary structure bound to the Fc region in case of an antibody. If one galactose molecule is found, it is called a G1 structure, if there is no galactose, a GO structure. A G2 glycosylation pattern was often found to improve the CDC of antibodies. Hence, it is preferred that at least 5%, 10%, 20%, 30%, 40%, 50%, 55%, 60%, 65%, 70%, 75%, 85%, 95% or even more than 100% higher amount of G2 structures are present in the protein/antibody composition produced. Suitable cell lines achieving a respective high G2 glycosylation pattern are described herein.

As a high overall galactosylation degree is often beneficial for the CDC of antibodies, it is often preferred to obtain 60%, 65%, 70%, 75%, 80%, 85%, 90%, 95% or even more than 95% of G2 and/or G1 structures on the total carbohydrate structures or on the carbohydrate structures at one particular glycosylation site of the antibody molecule.

G0 structures

Another possible characteristic of the antibody is that it has decreased amount of G0 structures, meaning that it has an amount of G0 structures on the total carbohydrate structures or on the carbohydrate structures at one particular glycosylation site of the antibody molecule of the antibody molecules in the antibody molecule composition which is at least 5% lower compared to the same amount of antibody molecules in at least one antibody molecule composition of the same antibody molecule isolated from ATCC No. CRL-9096 (CHOdhfr-) when expressed therein.

### Galalphal-3GaI

Another possible characteristic of the antibody is that it comprises no detectable terminal Galalpha1-3Gal.

The term “Galalpha1-3Gal” as used herein refers to galactose alpha(1-3) galactose. Without wishing to be bound by theory, a Galalpha1-3Gal glycosylation may be immunogenic in humans. This glycosylation characterizes a pattern, wherein a second galactose residue is linked in alpha 1 ,3 position to the first galactose residue, resulting in the highly immunogenic Galalpha 1-3 Gal disaccharide. By using immortalized human blood cells and in particular a host cell of human myeloid leukaemia origin, a respective disadvantageous glycosylation is avoided.

### Fucose

Another possible characteristic of the antibody is that it comprises an amount of fucose on the total carbohydrate structures or on the carbohydrate structures at one particular glycosylation site of the antibody molecule of said antibody molecules in the antibody molecule composition which is at least 5% less compared to the same amount of antibody molecules in at least one antibody molecule composition of the same antibody molecule isolated from ATCC No. CRL-9096 (CHOdhfr-) when expressed therein.

Fucose residues are found on different sites within the N-glycan tree so particularly:

alpha 1 ,6 linked to the GlcNAc residue proximal to the amino acid strain;

alpha 1 ,3 and alpha 1 ,4 linked to the antennary located GlcNAc residue;

alpha 1 ,2 linked to antennary located Gal residue.

On antibody attached N-glycans the vast majority of fucose residues is found 1 ,6 linked to the proximal GlcNAc residue (so called “core fucose”). It has been found that the absence of core fucose on the reducing end of the N-glycan attached to antibodies enhances the ADCC activity of antibodies by the factor 25 to 100. Due to this beneficial effect on ADCC, it is preferred that the amount of fucose is at least 5%, 10%, 15%, 20%, 30%, 40%, 50%, 60%, 70%, 80%, 90%, 100%, 150%, 200%, 250%, 300%, 400%, 500%, 1000%, 1500% or more than 2000% less compared to the same amount of protein molecules in at least one protein molecule composition of the same protein molecule isolated from ATCC No. CRL-9096 (CHOdhfr-) when expressed therein. In some embodiments, the antibody comprises at least 50%, preferably at least 60% and more preferably at least 70% carbohydrate structures of the total carbohydrate units or of at least one particular carbohydrate chain at a particular glycosylation site of an antibody molecule of the antibody molecules in said antibody molecule composition, lacking fucose.

### Bisecting GlcNAc

Another possible characteristic of the antibody is that it comprises at least one carbohydrate structure containing bisecting GlcNAc. Bisecting N-Acetylglucosamine (“bisGlcNAc” or “Bisecting GlcNAc”) is often found beta 1,4 attached to the central mannose residue of the tri-mannosyl core structure of the N-glycans found in antibodies. The presence of bisecting GlcNAc at the central mannose residue of the antibody Fc-N-glycan is thought to increase the ADCC activity of antibodies.

In some embodiments, the antibody comprises at least 2%, preferably at least 5%, more preferably at least 10% and most preferably at least 15% carbohydrate structures of the total carbohydrate units or of at least one particular carbohydrate chain at a particular glycosylation site of an antibody molecule of the antibody molecules in said antibody molecule composition which contains bisecting GlcNAc.

### Sialylation Pattern

Another possible characteristic of the antibody is that it has a sialylation pattern which is altered compared to the sialylation pattern of at least one antibody molecule composition of the same antibody molecule isolated from ATCC No. CRL-9096 (CHOdhfr-) when expressed therein.

The influence of the sialylation degree/pattern on the activity, half-live and bioavailability differs between different proteins/antibodies. Hence, it is beneficial to determine for each antibody molecule the optimized sialylation pattern in advance by using the screening method as described in WO2008028686, before establishing the production with the most suitable host cell, providing the desired glycosylation pattern. E.g. several publications exist reporting a negative impact of sialic acid residues present on the Fc glycan of antibodies on downstream effects, i.e. CDC and ADCC. However, it was found that a high silalylation prolongs the half-life of the sialylated molecules. Hence, depending on the antibody produced, a different sialylation pattern could be advantageous..

Preferably, said sialylation pattern is characterized by the following by at least one of the following characteristics:

E.g., the host cell can be selected such that it produces an antibody having a decreased sialylation degree with at least a 10% (preferably 15%, 20%, 25%, 30%, 35%, 40%, 45%, 50%, 55%, 60%, 65%, 70%, 75%, 80%, 85%, 90%, >95%) lower amount of sialic acids on the total carbohydrate structures or on the carbohydrate structures at one particular glycosylation site of the antibody molecule of the antibody molecules in the antibody molecule composition than the same amount of antibody molecules in at least one antibody molecule composition of the same antibody molecule isolated from ATCC No. CRL-9096 (CHOdhfr-) when expressed therein. E.g., the product may even comprise no detectable neuraminic acid (NeuNAc). Depending on the antibody produced, the presence of sialic acids and particularly NeuNAc may not contribute to the activity of the antibody. In these cases, it may be favourable to avoid sialic acids glycosylation in order make the product more homogeneous. This, as the NeuNAc glycosylation pattern can also vary in the resulting protein composition. This can cause difficulties in the regulatory approval of the product because the product is due to the varying NeuNAc content less homogeneous.

For antibodies which do not rely on the presence of a NeuNAc glycosylation for their activity, an avoidance of a NeuNAc glycosylation can be beneficial in order to increase homogeneity. However, “no detectable NeuNAc” does not necessarily mean that there is absolutely no NeuNAc present. Conversely, also embodiments are encompassed, which have a rather low degree of NeuNAc (e.g. 1 to 10%). One example of a respective protein is FSH.

According to one embodiment, the product has a decreased sialylation degree with a at least 15% (20%, 30%, 40%, 50%, 60%, 70%, 80%, 90%, 100%, 150%, 200%, 250%, 300%, 350%, 400%, 450%, >500%) lower amount of NeuNAc on the total carbohydrate structures or on the carbohydrate structures at one particular glycosylation site of the protein molecule of the protein molecules in said protein molecule composition than the same amount of protein molecules of at least one protein molecule composition of the same protein molecule isolated from ATCC No. CRL-9096 (CHOdhfr-) when expressed therein. This embodiment is beneficial in case a protein/antibody is supposed to be expressed, wherein the sialylation has a negative effect on the activity of the protein/antibody.

A respective glycosylation (absence or very low degree of sialic acid or particularly NeuNAc) can, e.g., be achieved by using sialylation deficient cells such as NM-F9 and NM-D4 in a serum-free medium.

According to a further embodiment, the product has an increased sialylation degree with an amount of NeuNAc on the total carbohydrate structures or on the carbohydrate structures at one particular glycosylation site of the antibody molecule of the antibody molecules in said antibody molecule composition which is at least a 15% (20%, 30%, 40%, 50%, 60%, 70%, 80%, 90%, 100%, 150%, 200%, 250%, 300%, 350%, 400%, 450% or more than 500%) higher compared to the same amount of protein molecules in at least one protein molecule composition of the same protein molecule isolated from ATCC No. CRL-9096 (CHOdhfr-) when expressed therein.

As was outlined above, a respectively increased degree of sialylation may provide a positive effect on the serum half-life of the protein by prolonging it. In these cases it is preferred to use a cell line which provides a higher degree of sialylation than is reached in ATCC No. CRL-9096 (CHOdhfr-) and which also provides a higher degree of sialylation than is reached in sialylation deficient cells (such as e.g. NM-F9 and NM-D4), wherein a precursor needs to be added in order to allow sialylation to occur. However, even if a respective precursor is added when growing these sialylation deficient cells, these cells usually only reach about 50 to 60% of the sialylation degree that is obtained with immortalized human blood cells having no genetic mutation/defect in the glycosylation machinery necessary for sialylation. Hence, for embodiments, wherein a higher degree of sialylation is aimed at, it is preferred to use cell lines capable of providing a respective high sialylation degree and not to use NM-F9 and NM-D4.

In some embodiments, the sialylation pattern is characterized by at least one of the following characteristics:
it comprises alpha2-6 linked NeuNAc; and/or
it has an increased sialylation degree with an amount of NeuNAc on the total carbohydrate structures or on the carbohydrate structures at one particular glycosylation site of the antibody molecule of the antibody molecules in said antibody molecule composition which is at least 15% higher compared to the same amount of antibody molecules in at least one antibody molecule composition of the same antibody molecule isolated from ATCC No. CRL-9096 (CHOdhfr-) when expressed therein, and/or
it comprises at least 20% more charged N-glycosidically linked carbohydrate chains of the total carbohydrate units or of at least one particular carbohydrate chain at a particular glycosylation site of the antibody molecule of the antibody molecules in said antibody molecule composition compared to the same amount of antibody molecules in at least one antibody molecule composition of the same antibody molecule isolated from ATCC No. CRL-9096 (CHOdhfr-) when expressed therein.

In this embodiment, the antibody comprises alpha2-6 linked NeuNAc. Additionally, alpha2-3 linked NeuNAc may be present to some extent. Regarding some proteins/antibodies the presence of a NeuNAc glycosylation is beneficial in particular regarding the half-life of the protein/antibody. To provide an alpha 2-6 linked NeuNAc is beneficial, because this glycosylation pattern resembles a human glycosylation pattern. Rodent cells usually provide an alpha2-3 linked NeuNAc. Also other existing human cell lines are not capable to provide a sufficient alpha 2-6 linked NeuNAc glycosylation.

Suitable cell lines to provide a respective glycosylation pattern are e.g. NM-H9D8 and NM-H9D8-E6.

Further, the antibody comprises at least 20% more charged N-glycosidically linked carbohydrate chains of the total carbohydrate units or of at least one particular carbohydrate chain at a particular glycosylation site of the protein molecule of said protein molecules in said protein molecule composition compared to the same amount of protein molecules in at least one protein molecule composition of the same protein molecule isolated from ATCC No. CRL-9096 (CHOdhfr-) when expressed therein.

The charge profile of a carbohydrate chain may also influence the properties and should thus be considered. Chemical groups which charge carbohydrate chains are e.g, sulphur groups or sialic acid.

In another embodiment, the antibody has an increased sialylation degree with an amount of NeuNAc on the total carbohydrate structures or on the carbohydrate structures at one particular glycosylation site of the antibody molecule of said antibody molecules in said antibody molecule composition which is at least 20%, preferably at least 30% higher compared to the same amount of antibody molecules in at least one antibody molecule composition of the same antibody molecule isolated from ATCC No. CRL-9096 (CHOdhfr-) when expressed therein.

Further embodiments

In a particular embodiment of the present invention, the host cell is selected to produce an antibody, comprising
at least 10% carbohydrate structures of the total carbohydrate units or of at least one particular carbohydrate chain at a particular glycosylation site of the antibody molecule of the antibody molecules in said antibody molecule composition, lacking fucose; and/or
at least 2% carbohydrate structures of the total carbohydrate units or of at least one particular carbohydrate chain at a particular glycosylation site of the antibody molecule of the antibody molecules in said antibody molecule composition which contains bisecting GlcNAc; and/or
more than 35% G2 structures on the total carbohydrate structures or on the carbohydrate structures at one particular glycosylation site of the antibody molecule in said antibody molecule composition; and/or
it comprises less than 22% G0 structures on the total carbohydrate structures or on the carbohydrate structures at one particular glycosylation site of the antibody molecule in said antibody molecule composition.

Preferred glycosylation combinations of the present invention are reflected in the following embodiment, according to which the host cell is selected to produce an antibody which
(a)
   comprises no detectable NeuGc
   comprises no detectable Galalpha1-3Gal
   comprises a galactosylation pattern as defined herein
   has a fucose content as defined herein
   comprises bisecGlcNAc
   comprises an increased amount of sialic acid compared to a antibody composition of the same antibody molecule when expressed in the cell line ATCC No. CRL-9096 (CHOdhfr-) or compared to a sialylation deficient cell line such as DSM ACC2606 (NM-F9) and DSM ACC2605 (NM-D4); or
      or which
(b)
   comprises no detectable NeuGc
   comprises no detectable Galalpha1-3Gal
   comprises a galactosylation pattern as defined herein
   has a fucose content as defined herein
   comprises bisecGlcNAc
   comprises 2-6 NeuNAc.

In some embodiments, the antibody is envisaged to
comprise no detectable NeuGc;
comprise a2,6-linked NeuNAc; and
have an increased sialylation degree with an amount of NeuNAc on the total carbohydrate structures or on the carbohydrate structures at one particular glycosylation site of the antibody molecule of said antibody molecules in said antibody molecule composition which is at least 15% higher compared to the same amount of antibody molecules in at least one antibody molecule composition of the same antibody molecule isolated from ATCC No. CRL-9096 (CHOdhfr-) when expressed therein.

In some embodiments, the antibody molecule has at least one of the following characteristics
it has a galactosylation degree of galactose, which is linked to GlcNAc, on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same antibody molecule isolated from ATCC No. CRL-9096 (CHOdhfr-) when expressed therein; and/or
it has an amount of GO structures on the total carbohydrate structures or on the carbohydrate structures at one particular glycosylation site of the antibody molecule of said antibody molecules in said antibody molecule composition which is at least 5% lower compared to the same amount of antibody molecules in at least one antibody molecule composition of the same antibody molecule isolated from ATCC No. CRL-9096 (CHOdhfr-) when expressed therein; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein.
it comprises at least 20% more charged N-glycosidically linked carbohydrate chains of the total carbohydrate units or of at least one particular carbohydrate chain at a particular glycosylation site of the antibody molecule of the antibody molecules in said antibody molecule composition compared to the same amount of antibody molecules in at least one antibody molecule composition of the same antibody molecule isolated from ATCC No. CRL-9096 (CHOdhfr-) when expressed therein, and/or
it comprises more than 35% G2 structures on the total carbohydrate structures or on the carbohydrate structures at one particular glycosylation site of an antibody molecule of the antibody molecules in said antibody composition; and/or
it comprises less than 22% GO structures on the total carbohydrate structures or on the carbohydrate structures at one particular glycosylation site of an antibody molecule of the antibody molecules in said antibody composition; and/or
it has an increased activity and/or increased yield compared to at least one antibody molecule composition of the same antibody molecule when expressed in the cell line ATCC No. CRL-9096 (CHOdhfr-); and/or
it has an improved homogeneity compared to at least one antibody molecule composition of the same antibody molecule when expressed in the cell line ATCC No. CRL-9096 (CHOdhfr-); and/or
it has an increased activity which is at least 10% higher than the activity of at least one antibody molecule composition from the same antibody molecule when expressed in the cell line ATCC No. CRL-9096 (CHOdhfr-); and/or
it has an increased Fc-mediated cellular cytotoxicity which is at least 2 to 5 times, such 2, 3, 4, 5 higher than the Fc-mediated cellular cytotoxicity of at least one antibody molecule composition from the same antibody molecule when expressed in the cell line ATCC No. CRL-9096 (CHOdhfr-); and/or it has an increased antigen mediated or Fc-mediated binding which is at least 50% higher than the binding of at least one antibody molecule composition from the same antibody molecule when expressed in the cell line ATCC No. CRL-9096 (CHOdhfr-); and/or - it has ADCC and/or CDC activity.

### Linker

The antibody can be linked to the drug by any means known in the art, for example by site-specific conjugation (e.g. by non-natural amino acids, engineered cysteine, a tag for enzyme-mediated conjugation, enzyme-mediated conjugation after deglycosylation, carbohydrate modification). Exemplary processes are e.g. disclosed in WO2009/099728. The person skilled in the art will be able to select suitable methods depending on the characteristics of the drug and the antibody, and factors such as pH, concentration, salt concentration, and co-solvents. In general, the antibody may be conjugated to the drug either directly or via a linker. Cytotoxic drugs can, e.g., be directly conjugated to antibodies through lysine or cysteine residues of an antibody. E.g., the antibody may be reduced with a reducing agent such as dithiothreitol (DTT) or tricarbonylethylphosphine (TCEP), under partial or total reducing conditions, to generate reactive cysteine thiol groups. The antibody can be subjected to denaturing conditions to reveal reactive nucleophilic groups such as lysine or cysteine. Enzymes may be covalently bound to antibodies by recombinant DNA techniques well known in the art.

The term “linker” denotes a chemical moiety comprising a covalent bond or a chain of atoms that covalently attaches an antibody to a drug moiety. In various embodiments, a linker is specified as L. Linkers include, but are not limited to, a divalent radical such as an alkyldiyl, an aryldiyl, a heteroaryldiyl, moieties such as: —(CR2)nO(CR2)n—, repeating units of alkyloxy (e.g. polyethylenoxy, PEG, polymethyleneoxy) and alkylamino (e.g. polyethyleneamino, Jeffamine(™)); and diacid ester and amides including maleimide, succinate, succinamide, diglycolate, malonate, and caproamide.

### Drug

The term “drug moiety” or “payload” as used herein refers to a chemical moiety that is conjugated to an antibody or antigen binding fragment of the invention, and can include any therapeutic or diagnostic agent, for example, an anti-cancer, anti-inflammatory, anti-infective (e.g., anti-fungal, antibacterial, anti-parasitic, anti-viral), or an anesthetic agent. The antibody drug conjugate (ADC) of the present invention is envisaged to comprise one or more drugs, preferably drugs for cancer therapy, including but not limited to, cytotoxins, chemotherapeutic agents, toxins such as small molecule toxins or enzymatically active toxins of bacterial, fungal, plant or animal origin, including synthetic analogs and derivatives thereof. In particular, antibodies of the present invention may be conjugated to a growth inhibitory agent or cytotoxic agent such as a toxin, including, for example, auristatin, auristatin E (AE), maytansine, maytansinoid, dolostatin, DM1, DM3, DM4, monomethylauristatin (MMAE), MMAF or calicheamicin, an antibiotic, a nucleolytic enzyme (e.g., a ribonuclease or a DNA endonuclease such as a deoxyribonuclease), DNase, BCNU, streptozocin, vincristine and 5-fluorouracil, the LL-E33288 complex, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of Lu), enzymatically active toxins and fragments thereof (e.g. diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes), prodrug-activating enzymes (e.g. alkaline phosphatases, arylsulfatases, cytosine deaminase, proteases, such as serratia protease, thermolysin, subtilisin, carboxypeptidases and cathepsins (such as cathepsins B and L), D-alanylcarboxypeptidases, carbohydrate-cleaving enzymes (e.g. β-galactosidase, neuraminidase, β-lactamase, penicillin amidases, such as penicillin V amidase and penicillin G amidase, alkylating agents or agents with an alkylating action, such as cyclophosphamide (CTX; e.g. Cytoxan®), chlorambucil (CHL; e.g. Leukeran®), cisplatin (CisP; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6 MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and paclitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin and other folic acid derivatives, arnifostine (e.g. Ethyol®), dactinomycin, mechlorethamine (nitrogen mustard), streptozocin, cyclophosphamide, lomustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, pyrrolobenzodiazepine (PBD) or carbon monoxide.

Other drugs are peptides, RNA, DNA or cytokines. DNA and RNA may have inhibitory function on target mRNA or DNA. Peptides may have a cytotoxic effect.

Particularly preferred drugs in the context of the present invention are cytotoxins, such as microtubule inhibitors or DNA-damaging agents, or the cytotoxic agents described herein.

For the purpose of the invention the compounds as described herein also includes derivatives and pharmaceutically acceptable salt(s) thereof. The phrase “pharmaceutically or cosmetically acceptable salt(s)”, as used herein, means those salts of compounds of the invention that are safe and effective for the desired administration form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Host Cell

According to the invention, the term “host cell” relates to any cell which can be transformed or transfected with an exogenous nucleic acid. The term “host cell” generally comprises prokaryotic (e.g. E. coli) or eukaryotic cells (e.g. mammalian cells, in particular human cells, yeast cells and insect cells). Any host cell can be used in the methods of the invention as long as it is able to produce antibodies having the desired characteristics described elsewhere herein. Particular preference is given to mammalian cells such as cells from humans, mice, hamsters, pigs, goats, or primates. The cells may be derived from a multiplicity of tissue types and comprise primary cells and cell lines. Preferably, the host cell is a human cell, in particular an immortalized human cell, preferably an immortalized human blood cell such as an immortalized human myeloid cell or an immortalized human myeloid leukemia cell. Furthermore, the host cell may also be an immortalized human tumor cell. Preferably, the host cell is selected from the group consisting of NM-F9 [DSM ACC2606], NM-D4 [DSM ACC2605], GT-2X [DSM ACC2858], NM-H9, NM-E-2F9, NM-C-2F5, NM-H9D8[DSM ACC2806], NM-H9D8-E6 [DSM ACC2807], NM-H9D8-E6Q12 [DSM ACC2856], GT-5s [DSM ACC 3078] or a cell or cell line derived therefrom. A nucleic acid may be present in the host cell in the form of a single copy or of two or more copies and, In some embodiments, is expressed in the host cell.

### ADC

The present invention further also relates to an ADC obtainable by the method as described herein for use in a method of treatment of cancer in a patient. The term “patient” means according to the invention a human being, a non-human primate or another animal, in particular a mammal such as a cow, horse, pig, sheep, goat, dog, cat or a rodent such as a mouse and rat. In a particularly preferred embodiment, the patient is a human being. Except when noted, the terms “patient” or “subject” are used herein interchangeably The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). The exact amount dose will depend on the purpose of the treatment, and will be ascertainable by one skilled in the art using known techniques. As is known in the art and described above, adjustments for age, body weight, general health, sex, diet, drug interaction and the severity of the condition may be necessary, and will be ascertainable with routine experimentation by those skilled in the art. In some embodiments, a therapeutically acceptable amount does not induce or cause undesirable side effects. In some embodiments, a therapeutically acceptable amount induces or causes side effects but only those that are acceptable by the healthcare providers in view of a patient's condition. A therapeutically acceptable amount can be determined by first administering a low dose, and then incrementally increasing that dose until the desired effect is achieved. A “prophylactically effective dosage,” and a “therapeutically effective dosage,” of the molecules of the invention can prevent the onset of, or result in a decrease in severity of, respectively, disease symptoms, including symptoms associated with cancer. An ADC of the present invention is preferably administered in a therapeutically acceptable amount (or therapeutically effective dose). Similarly, an ADC of the present invention is administered preferably in prophylactically effective dosage (or therapeutically effective dosage).

The method comprises administering said ADC at doses which are lower than doses for an ADC which was not obtained from a host cell having at least one of the glycosylation characteristics as defined above. The embodiments and characteristics described in the context of the method of the invention also apply to the ADC of the invention, mutatis mutandis.

### Cancer

In some embodiments, the antibody of said ADC is directed against a molecule on the surface of a cancer cell. Said cancer, is, In some embodiments, characterized by a solid tumor, which may be a breast cancer tumor. However, said cancer may also be a blood-borne cancer.

The term “cancer” according to the invention in particular comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. The term cancer according to the invention also comprises cancer metastases.

By “tumor” is meant a group of cells or tissue that is formed by misregulated cellular proliferation, in particular cancer. Tumors may show partial or complete lack of structural organization and functional coordination with the normal tissue, and usually form a distinct mass of tissue, which may be either benign or malignant. In particular, the term “tumor” refers to a malignant tumor. According to one embodiment, the term “tumor” or “tumor cell” also refers to non-solid cancers and cells of non-solid cancers such as leukemia cells. According to another embodiment, respective non-solid cancers or cells thereof are not encompassed by the terms “tumor” and “tumor cell”.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

### Other Medical Conditions

ADCs of the present invention can also be used for prophylactic and/or therapeutic treatment of diseases, such as leukemia, neutropenia, cytopenia, cancer, bone marrow transplantation, diseases of hematopoietic systems, infertility and autoimmune diseases.

Accordingly, in some embodiments, the antibody of said ADC is directed against a molecule on the surface of a cell such as an immune cell, blood cell, or bone marrow cell, or a cell infected with a bacterium, virus or parasite.

### Particular Antibodies

In some embodiments, the antibody of the ADC is an immune-modulatory antibody, an antibody against ganglioside GD3, antibodies against human interleukin-5 receptor alphachain, antibodies against HER2, antibodies against CC chemokine receptor 4, antibodies against CD20, antibodies against CD4, CD7, CD8, CD22 CD30, CD33, CD52, CD19, CD138, CD22, CD70, CD74, CD56, GPNMB, PSMA, SLC44A4, CA6, CA-IX, mesothelin, CD66e/CEACAM5, Nectin-4, antibodies against neuroblastoma, antibodies against MUC1, antibodies against TA-MUC1, antibodies against Lewis Y, antibodies against epidermal growth factor receptors, such as HER1, HER2, HER3, HER4, antibodies against immune checkpoint proteins, such as PD-1, PD-L1, CTLA-1; in particular an antibody selected from the group consisting of Pankomab, Muromomab, Daclizumab, Basiliximab, Abciximab, Rituximab, Herceptin, Gemtuzumab, Alemtuzumab, Ibritumomab, Cetuximab, Bevacizumab, Tositumomab, Pavlizumab, Infliximab, Eculizumab, Epratuzumab, Omalizumab, Efalizumab, Adalimumab, OKT3, anti-CC chemokine receptor 4 antibody KM2160, and anti-neuroblastoma antibody chCE7.

In some embodiments, the antibody of the ADC of the invention comprises
(i) a heavy chain variable region comprising a CDR1 having the amino acid sequence of SEQ ID NO: 1 , a CDR2 having the amino acid sequence of SEQ ID NO: 2, and a CDR3 having the amino acid sequence of SEQ ID NO: 3; and
(ii) a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6.

In some embodiments, the antibody of the ADC comprises
(i) a heavy chain variable region comprising the amino acid sequence of SEQ ID NO: 7 or an amino acid sequence which is at least 80% identical thereto;
(ii) a light chain variable region comprising the amino acid sequence of SEQ ID NO: 8 or an amino acid sequence which is at least 80% identical thereto.

In some embodiments, the antibody of the ADC comprises
(i) a heavy chain comprising the amino acid sequence of SEQ ID NO: 9 or an amino acid sequence which is at least 80% identical thereto;
(ii) a light chain variable region comprising the amino acid sequence of SEQ ID NO: 10 or an amino acid sequence which is at least 80% identical thereto.

In some embodiments, the antibody of the ADC comprises one or more of the CDRs shown in
(i) SEQ ID NO: 11, 12, 23, 24, 25, 26, 27, 28, 29, or 30; SEQ ID NO: 13, 14, 31, 32, or 33 and SEQ ID NO: 15 or 16: and/or
(ii) SEQ ID NO: 17, 18, 34, 35, 36, 37, 38, or 39; SEQ ID NO: 19 or 20 and SEQ ID NO: 21, 22, 40, or 41.

In some embodiments, the antibody of the ADC comprises one or more of the CDRs shown in
(i) SEQ ID NO: 42, 55, 56, 57, or 58; SEQ ID NO: 43, 44, 59, 60, 61, 62, 63, 64, 65, 66, 67, or 68; and SEQ ID NO: 45, 46, or 47; and/or
(ii) SEQ ID NO: 48, 49, 50, 69, 70, 71, 72, 73, 74, 75, 76, 77, 78, 79, 80, 81, 82, 83, 84, 85, or 86; SEQ ID NO: 51 or 52; and SEQ ID NO: 53 or 54.

### Function

The ADC which is obtained as described herein is envisaged to have an improved safety profile and/or efficacy. When used herein, the term “safety” or “safety profile” as used herein defines the administration of an ADC of the present invention to a patient essentially without, ideally without inducing severe adverse events directly after administration (local tolerance) and during a longer period of application of the drug. Put differently, a safety profile advantageously aims at avoiding or ameliorating a noxious and/or unintended side effect of a drug, here an ADC, including avoiding a lack of efficacy.

“Safety” can be evaluated e.g. at regular intervals during the treatment and follow-up period. Measurements include clinical evaluation, e.g. organ manifestations, and screening of laboratory abnormalities. Clinical evaluation may be carried out and deviating to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v3.0 (CTCAE). Laboratory parameters which may be tested include for instance haematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events may also be examined by histopathological and/or histochemical methods.

“Improving the safety profile” or “improved safety profile” means that the means and methods of the present invention allow the improvement of the safety profile of an ADC of the present invention. In particular, an improvement results preferably in a reduction of the dose of an ADC of the present invention. Thus, due to the reduction of the dose (a) risk(s) for a patient to suffer from or acquire an undesired side effect or at the least suffer only from side effects that are acceptable by the healthcare providers in view of a patient's condition are believed to be lowered. Without being bound by theory, it is assumed that ADCs of the present invention can be administered below doses of ADCs that are not obtained from a host cell having at least one of the glycosylation characteristics as described herein. Put it differently, ADCs, the antibody part thereof does not have the glycosylation characteristics as described for antibodies herein, do likely have to be administered in higher doses than ADCs of the present invention in order to have the ADC mediate enhanced Fc receptor binding, particularly mediate ADCC. To this end, ADCs of the present invention are assumed to mediate enhanced Fc receptor binding, particularly to mediate ADCC at amounts at which ADCs that are not obtained or produced from a host cell having at least one of the glycosylation characteristics as described herein will essentially not, preferably not mediate enhanced Fc receptor binding, particularly mediate ADCC. Accordingly, ADCs of the present invention are advantageous, since they are assumed to mediate enhanced Fc receptor binding, particularly mediate ADCC at amounts or concentrations at which other ADCs, i.e. ADCs that are not obtained from a host cell having at least one of the glycosylation characteristics as described herein will not mediate enhanced Fc receptor binding, particularly mediate ADCC.

“Efficacy” means that ADCs of the present invention have an enhanced Fc receptor binding, particularly Fc gamma receptor III binding and/or enhanced ADCC in comparison to ADCs which do not have at least one of the characteristics of the ADCs of the present invention as described herein and/or which have not been produced by a host cell of the present invention having at least one of the characteristics as described herein.

For the approximation of the magnitude of the ADCC enhancement of two antibodies, concentration curves of said two antibodies are measured in parallel on the same plate for their target. Curve fitting is performed for both antibodies separately using, for example advantageously a four-parameter (4PL) logistic plot calculated by GraphPad Prism 5 software version 5.01. Specific lysis values at certain antibody concentrations or the antibody concentration corresponding to certain specific lysis values are interpolated from the curves.

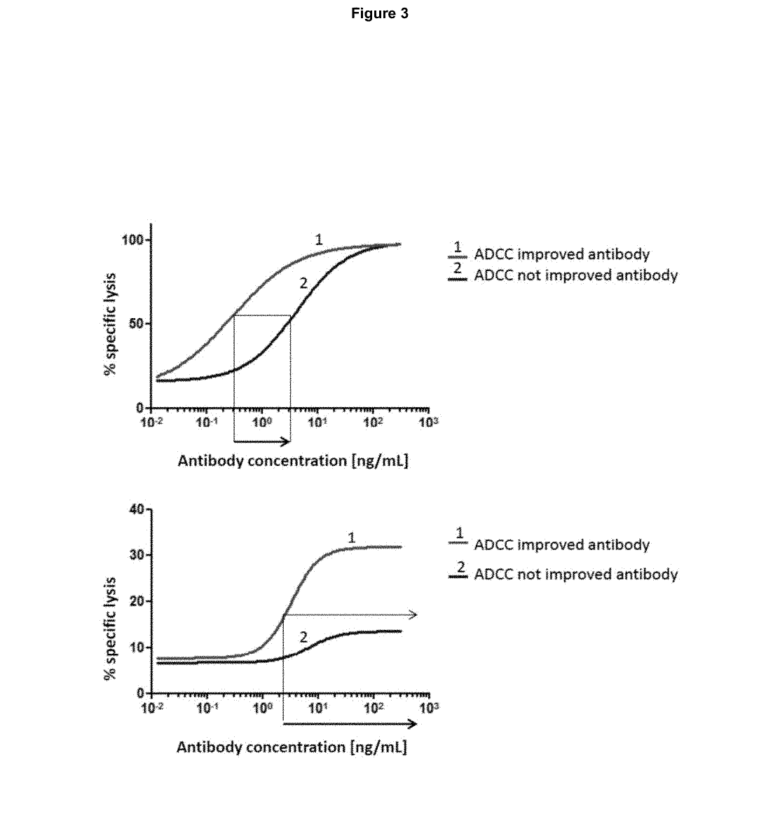

Improvement factor was calculated by comparing the antibody concentration necessary to achieve 50% of maximal lysis of the assumed improved antibody molecule, whereby the necessary concentration of the assumed not-improved antibody was divided by the necessary concentration of the assumed improved antibody. The improvement factor can reach an infinite value if the assumed not-improved antibody does not achieve 50% of the specific lysis of the improved antibody at all (see FIG. 3 for an illustration of the afore-described determination of ADCC enhancement).

In some embodiments, the ADC has enhanced Fc receptor (FcR) binding resulting in enhanced immunological effector functions of said ADC. Said enhanced FcR binding is envisaged to be enhanced to the extent such that said ADC mediates enhanced immunological effector functions at doses at which essentially no immunological effector functions are mediated in comparison to said ADC without enhanced FcR binding. In some embodiments, said enhanced FcR binding is enhanced Fc gamma receptor binding, particularly Fc gamma receptor III binding, and/or mediates enhanced antibody dependent cell cytotoxicity (ADCC).

The therapeutic efficacy of antibodies or ADCs - in addition to their circulation half-life —in many cases depends on the induction of cytotoxic effects, in particular antibody—dependent cell-mediated cytotoxicity activity (“ADCC”), against the target cells bound by the antibody. Therefore, increasing in particular the ADCC activity of an antibody increases the therapeutic value thereof. For example, the same amount of antibodies administered to a patient will achieve a much higher therapeutic benefit when using antibodies optimized for their ADCC activity. Furthermore, for achieving the same therapeutic effect, a much lower amount of such antibodies has to be administered. As discussed herein, also the increase of the antibody's circulation half-life results in an enhanced therapeutic effect. Thus, a combination of both is also envisaged in the context of the present invention.

Without wishing to be bound by theory, the enhanced immunological effector functions are thought to be achieved by the optimized glycosylation pattern, in particular the optimized glycosylation pattern at the Fc part of the antibodies. For example, the ADCC activity of antibodies of the IgG type is mediated by binding of the antibody to Fc gamma-receptors, in particular Fc gamma RIII, via its Fc part. Fc gamma RIII is expressed on natural killer (NK) cells and macrophages and upon activation by an antibody induces the release of cytokines and cytotoxic granules which results in apoptosis of the target cell bound by the antibody. The binding affinity of the antibody to the Fc gamma-receptor is influenced by the carbohydrates attached to the glycosylation sites at the Fc part of the antibody. Therefore, optimization of the glycosylation pattern on the Fc part of an antibody will result in a stronger Fc gamma RIII-binding and thus, in an enhanced ADCC activity.

### Chemical Modification

The antibody of the ADC according to the invention may be chemically modified. Generally, all kind of modifications are envisaged by the present invention as long as they do abolish the advantageous capabilities of the antibody, i.e. the chemically modified compounds of the invention should preferably have capabilities which are comparable to the capabilities of the compounds which were evaluated in the appended examples.. Possible chemical modifications of the antibody include acylation or acetylation of the amino-terminal end or amidation or esterification of the carboxy-terminal end or, alternatively, on both. The modifications may also affect the amino group in the side chain of lysine or the hydroxyl group of threonine. Other suitable modifications include, e.g., extension of an amino group with polypeptide chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e.g., methylation, propylation, butylation), arylation, and etherification may be possible and are also envisaged.

### Co-administration

The ADC of the invention may be co-administered with other agents, in particular anticancer drugs, or compounds that enhance the effects of such agents. Co-administration comprises sequential and simultaneous administration. Suitable anticancer drugs include, e.g., the drugs described as drug conjugates in the context of the ADC.

### Pharmaceutical Composition

The ADC of the invention may also be present in the form of a pharmaceutical composition.

The term “pharmaceutical composition” particularly refers to a composition suitable for administering to a human or animal, i.e., a composition containing components which are pharmaceutically acceptable. Preferably, a pharmaceutical composition comprises an ADC together with a carrier, diluent or pharmaceutical excipient such as buffer, preservative and tonicity modifier. Pharmaceutical compositions of the invention comprise a therapeutically effective amount of ADC and can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for topical or oral administration.

Preferably a fluid composition is used, more preferably an aqueous composition. It preferably further comprises a solvent such as water, a buffer for adjusting and maintaining the pH value, and optionally further agents for stabilizing the ADC or preventing degradation of the ADC. The composition preferably comprises a reasonable amount of ADC, in particular at least 1 fmol, preferably at least 1 μmol, at least 1 nmol or at least 1 μmol of the antibody. It may additionally comprise further antibodies or ADCs.

A variety of routes are applicable for administration of the pharmaceutical composition, including, but not limited to, orally, topically, transdermally, subcutaneously, intravenously, intraperitoneally, intramuscularly or intraocularly. However, any other route may readily be chosen by the person skilled in the art if desired.

### EXAMPLES

The present examples were made with an antibody that is, in accordance with the teaching of the present invention, part of an ADC and are thus representative for an ADC. Indeed, there is no doubt that an ADC will work, however, the clue of the present invention is the additional feature that is provided by an antibody obtainable from a host cell having at least one of the characteristics as described herein, i.e., enhanced Fc receptor binding, preferably Fc gamma receptor III binding and/or enhanced ADCC.

Similarly, the present examples were made with an antibody that is directed to Her2. However, this antibody merely shows exemplarily that the general principle, i.e., applying a host cell having the characteristics as described herein, will apply to each and every antibody, since glycosylation is a process that does—in a host cell of the present invention—automatically occur when such a host cell expresses an antibody. Hence, the antibody used in the examples is in fact merely representative of each and every antibody.

### Example 1

### Glycooptimized Antibody against Her2 is fully Comparable with Herceptin in Antigen Binding, Specificity, Affinity and Fv-mediated Anti-tumor Activity

### Tumor Cell Binding

Several HER2 positive cell lines were analyzed by flow cytometry in order to compare the binding properties of glycooptimized antibody against Her2 (TrasGEX) and Herceptin. TrasGEx is characterized by any one of SEQ ID NOs: 1-10.

Briefly, target cells were harvested and incubated with TrasGEX or Herceptin (Roche) at different concentrations. Cells were washed and incubated with a secondary Cy3-conjugated anti-human IgG antibody at 4° C. in the dark. Cells were washed and analyzed in a flow cytometer FACS Canto II (Becton Dickinson). Viable cells were gated based on their scatter properties and the percentage of positive cells was calculated using the FACSDiva Software (Becton Dickinson).

### Inhibition of Proliferation

Binding of Herceptin to the extracellular domain of HER2 results in the inhibition of proliferation of tumor cells (Brockhoff et al., Cell Prolif 2007, 40: 488-507; Spiridon et al., 2002, Clinical Cancer Research 2002, 8: 1720-1730). In order to analyze this mechanism of action for TrasGEX, proliferation of SK-BR-3 cells (human breast carcinoma cell line) was measured in an MTT assay with different concentrations (0.1-100 μg/mL) of TrasGEX or Herceptin. The MTT assay is a non-radioactive assay based on the cleavage of the soluble yellow tetrazolium salt MTT (3-[4,5-Dimethylthiazol-2-yl]-2,5-diphenyltetrazolium bromide; Thiazolyl Blue) by mitochondrial dehydrogenases of viable cells. This results in the formation of a purple formazan, which can be measured in an ELISA reader at 570 nm. The absorbance signal is a direct measure for viable cells in the culture.

As a positive control, proliferation was completely inhibited by addition of taxol. Human IgG1 or medium alone served as negative controls. Briefly, SK-BR-3 cells were grown for 2 days in 96-well flat bottom plates. TrasGEX, Herceptin and control substances (hIgG1 and Taxol (20 nM)) were added and the plates were incubated for another 4-6 days at 37° C. in a humidified CO₂ incubator. The supernatant was completely removed and MTT was added. Cells were incubated for 2 hours with MTT at 37° C. in a humidified CO₂ incubator. The supernatant was removed and cells were lysed using HCI and 2-propanol containing lysis buffer for 1 h at room temperature in the dark. Absorbance at 570 nm/630 nm was measured in a plate reader Infinite F200 (Tecan Austria GmbH).

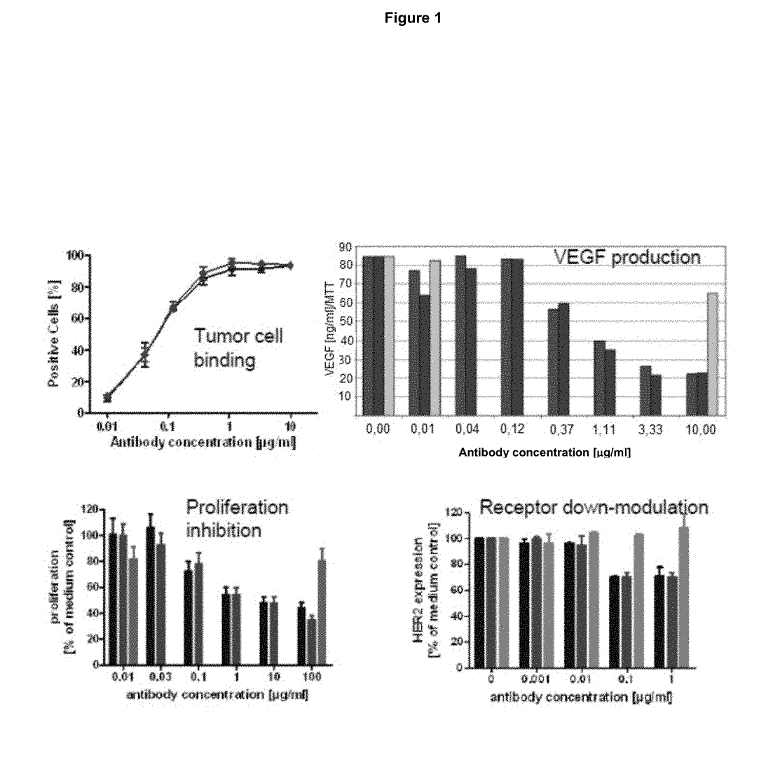

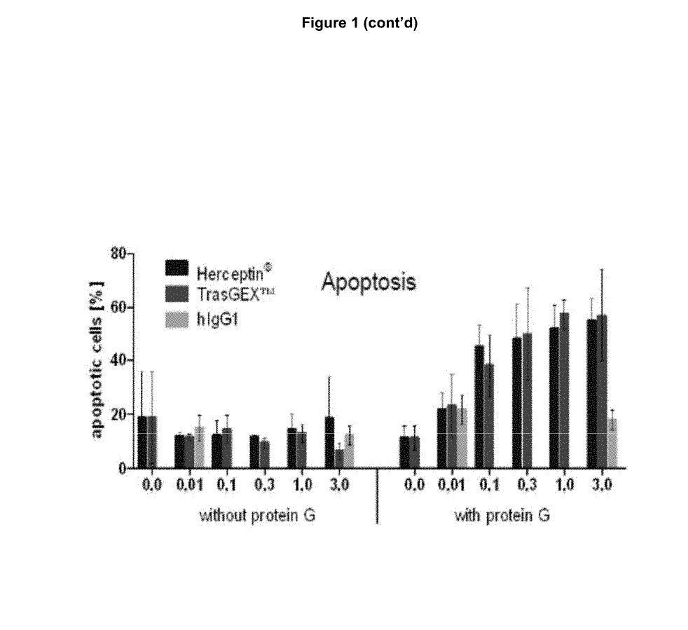

FIG. 1 shows mean results of three independent experiments performed with TrasGEX) and Herceptin. Proliferation after 4 days of incubation with the antibodies was calculated relative to the proliferation in the medium control. The positive control Taxol (20 nM) resulted in maximal proliferation inhibition (only 6% proliferation compared to the medium control; data not shown). TrasGEX and Herceptin induced a concentration-dependent inhibition of proliferation of SK-BR-3 cells. At an antibody concentration of 100 μg/mL, proliferation was reduced by more than 50%. Using Bonferroni post-tests, there was no significant difference in the proliferation inhibition induced by TrasGEX and Herceptin.

### VEGF Production

Malignant tumors secrete angiogenic factors regulating the tumors own blood supply by neoangiogenesis. Herceptin treatment of experimental human breast tumors in mice was shown to induce normalization and regression of the tumor vasculature. Diameter and volume as well as vascular permeability of tumor blood vessels were reduced (Izumi et al., 2002, Nature 2002, 416: 279-280). Breast cancer cell lines and primary breast cancers can express vascular endothelial growth factor (VEGF). Increased VEGF expression in breast cancers is associated with tumor progression. HER2 signaling is known to regulate the production of VEGF in human breast cancer cell lines (Izumi et al., 2002, loc. cit.; Emlet et al., 2007, Mol Cancer Ther 2007, 6(10): 2664-2674). There is evidence for a reduction of VEGF production in human breast cancer cell lines by Herceptin in vitro (Emlet et al., 2007, loc. cit.). Therefore, production of VEGF was studied in a human breast cancer cell line after incubation with TrasGEX or Herceptin.

Briefly, HER2 positive target cells of the human cell line BT474 were plated into 96-well flat bottom plates and incubated overnight in a humidified CO₂ incubator. TrasGEX, Herceptin or hIgG1 as a control were added at concentrations of 0.01 to 10 μg/mL. At day 3, cells were fed with media supplemented with the antibodies. At day 6, culture supernatant was collected and the remaining cells were analyzed for viable cells using MTT assay as described for proliferation. Cell supernatant was analyzed for its VEGF content using a commercially available Human VEGF ELISA-Kit (cell sciences). In order to correct for the reduction in cell numbers by TrasGEX or Herceptin induced proliferation inhibition, measured mean VEGF concentration was divided by the mean ODs from MTT assays as a measure of viable cell numbers.

FIG. 1 shows the results of an experiment performed with BT474 cells after 6 days of incubation with TrasGEX or Herceptin. There was a concentration-dependent reduction in VEGF production observed in TrasGEX- and Herceptin-treated BT474 cell cultures. The observed effects were comparable for TrasGEX and Herceptin.

### Receptor Down-modulation

In order to analyze the capacity of TrasGEX to down-modulate the HER2 receptor expression in a similar way as Herceptin, a receptor down-modulation study was performed comparing this mechanism of action for both antibodies. HER2 receptor down-modulation was analyzed by flow cytometry.

Briefly, ZR-75-1 cells were seeded into 96 well flat bottom plates and incubated for one day at 37° C. in a CO2 incubator. TrasGEX, Herceptin, or hIgG1 as a negative control at different concentrations were added. The plates were incubated for 3 to 4 days at 37° C. in a CO2 incubator.

ZR-75-1 cells were harvested and stained with a FITC-conjugated anti-human HER2 antibody (BMS120F1, eBioscience, Bender Medsystems) recognizing an epitope different from that bound by TrasGEX and Herceptin. Using this antibody, staining of the HER2 receptor is possible despite the presence of TrasGEX or Herceptin. BMS120F1 positive cells were analyzed by flow cytometry at a BD FACS Canto ll flow cytometer using BD FACSDiva Software.

FIG. 1 shows the mean results of two independent assays using ZR-75-1 cells after 4 days of incubation with TrasGEX, Herceptin or hIgG1. HER2 receptor expression is given as the percentage of HER2 positive cells of the medium control. It could be shown that the HER2 expression in the presence of TrasGEX or Herceptin was reduced by about 30% compared to the medium control. The human IgG1 isotype control did not result in a reduced HER2 receptor expression.

### Induction of Apoptosis

Induction of apoptosis is a further mechanism by which antibodies can mediate anti-tumor activity. While direct induction of apoptosis by monomeric antibodies is often ineffective (as seen for rituximab, Zhang et al., 2005, Clin Cancer Res 2005, 11(16): 5971-5980) cross-linking of the antibody by anti-human immunoglobulin or protein G evokes this mechanism of action. In vivo, cross-linking of the antibody can be induced by Fc-receptor-bearing cells, e.g. neutrophils were shown as potential physiological cross-linkers thereby augmenting rituximab-induced apoptosis (Nakagawa et al., 2010, Leukemia Research 2010, 34: 666-671).

In order to study this potential mode of action, we analyzed the induction of apoptosis by TrasGEX and Herceptin without and with cross-linking by protein G on the tumor cell line BT474. As a marker for induction of apoptosis, we analyzed the activation of caspase-3 using the BD PE Active Caspase-3 Apoptosis Kit. Briefly, tumor cell lines were cultured in medium containing 1% FCS for one day prior to the assay. Cells were seeded into 48 well plates and incubated at 37° C. in a CO₂ incubator overnight. TrasGEX, Herceptin or hIgG1 as a negative control at different concentrations were added. For samples with cross-linking, protein G at a final concentration of 2 μg/mL was added. The plates were incubated for 4 to 48 h at 37° C. in a CO₂ incubator.

Cells (both adherent and non-adherent cells) were harvested, permeabilized, fixed and stained for active caspase-3 according to manufacturer's protocol. Active caspase-3-positive (apoptotic) cells were analyzed by flow cytometry at a BD FACS Canto II flow cytometer using BD FACSDiva™ Software.

FIG. 1 shows the results of an active caspase-3 apoptosis assay using BT474 cells. After cross-linking by protein G, TrasGEX induced strong concentration-dependent apoptosis in BT474 cells. Apoptosis induction was comparable between TrasGEX and Herceptin. No apoptosis was induced by TrasGEX or Herceptin in the absence of the cross-linker protein G under the conditions analyzed.

### Example 2

### Glycooptimized Antibody having Highly Improved ADCC for Treatment of all Breast Cancer Patient Subgroups, Particularly with Low Her2 Expressing Tumors

The assay was performed as a europium release assay. Briefly, HER2-positive target cell lines (SK-BR-3; MCF-7) were loaded with europium (Eu³+) by electroporation and incubated with thawed primary human peripheral blood mononuclear cells (PBMCs, effector cells, stored in liquid nitrogen) at an effector-to-target cell ratio (E:T ratio) of 50:1 in the presence of TrasGEX, Herceptin or human control antibodies (hIgG1) at different concentrations for 5 hours. Europium release into the supernatant (indicating antibody-mediated cell death) was quantified using a fluorescence plate reader Infinite F200 (Tecan Austria GmbH). Maximal release was achieved by incubation of target cells with Triton X-100 and spontaneous release was measured in samples containing only target cells alone. Specific cytotoxicity was calculated as:

$$\% specific lysis = \frac{experimental release - spontaneous release}{maximal release - spontaneouse release} \times 100$$

For the approximation of the magnitude of the ADCC enhancement of TrasGEX compared to Herceptin, concentration curves of TrasGEX and Herceptin were measured in parallel on the same plate for each donor. Curve fitting was performed for both antibodies separately using a four-parameter (4PL) logistic plot calculated by GraphPad Prism 5 software version 5.01. Specific lysis values at certain antibody concentrations or the antibody concentration corresponding to certain specific lysis values were interpolated from the curves.

Improvement factor was calculated by comparing the antibody concentration necessary to achieve 50% of maximal lysis of the improved antibody molecule, whereby the necessary concentration of the not improved antibody was divided by the necessary concentration of the improved antibody. The improvement factor can reach an infinite value if the not improved antibody does not achieve 50% of the specific lysis of the improved antibody at all (see FIG. 3).

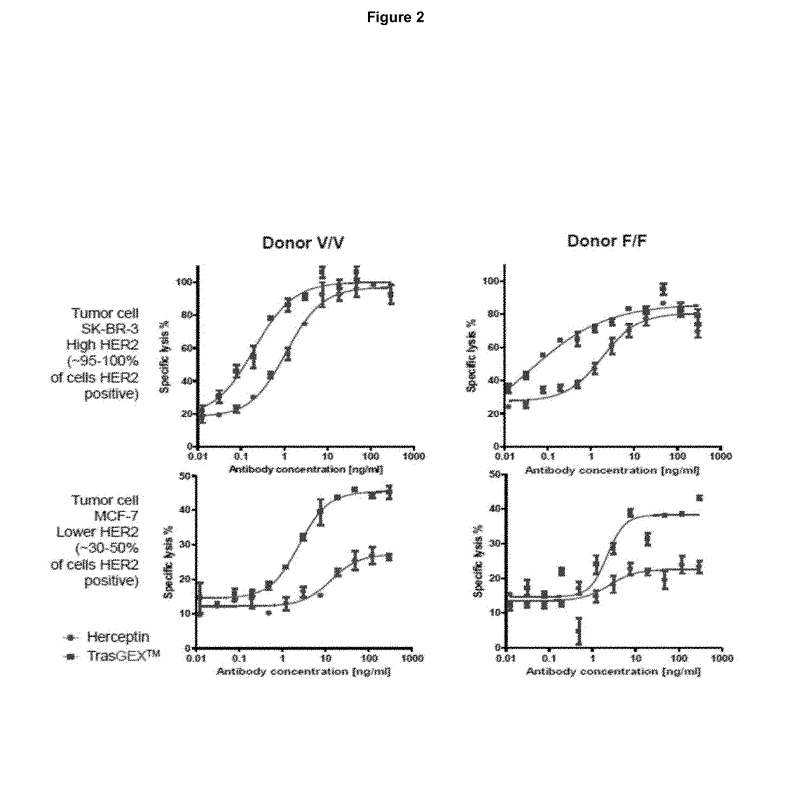

It can be seen in FIG. 2 that TrasGEX shows an improved ADCC.

### Example 3

### Internalization of a Glycooptimized Antibody against Her2 into Acidic Compartments of Cancer Cells

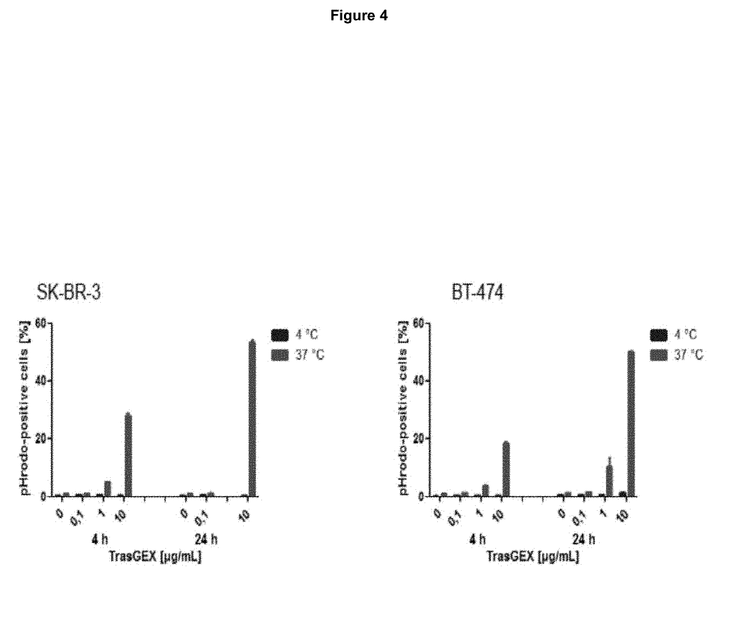

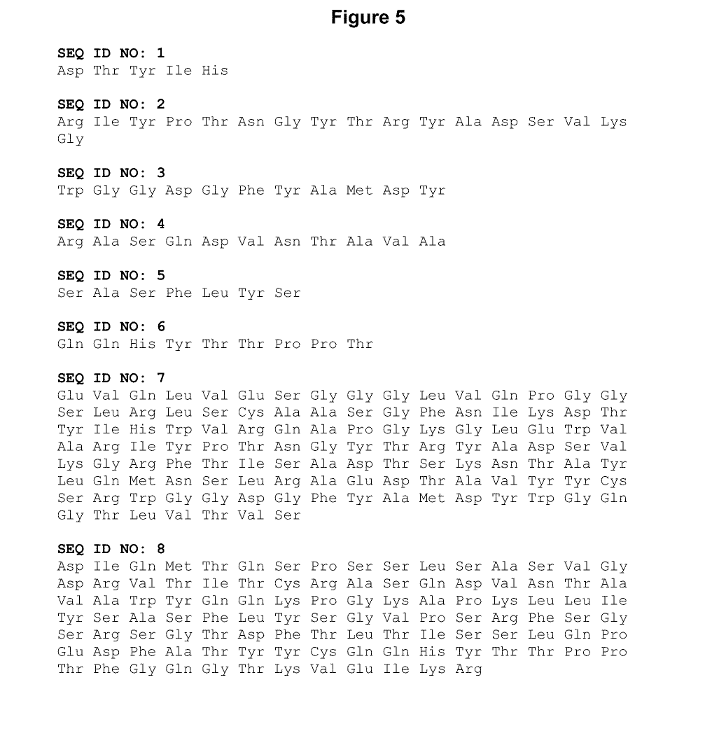

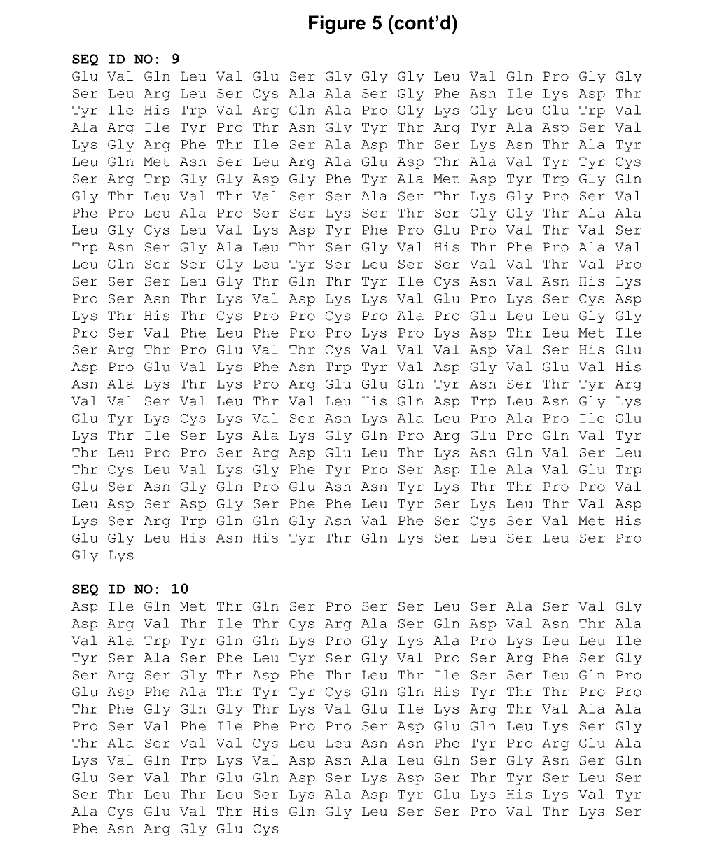

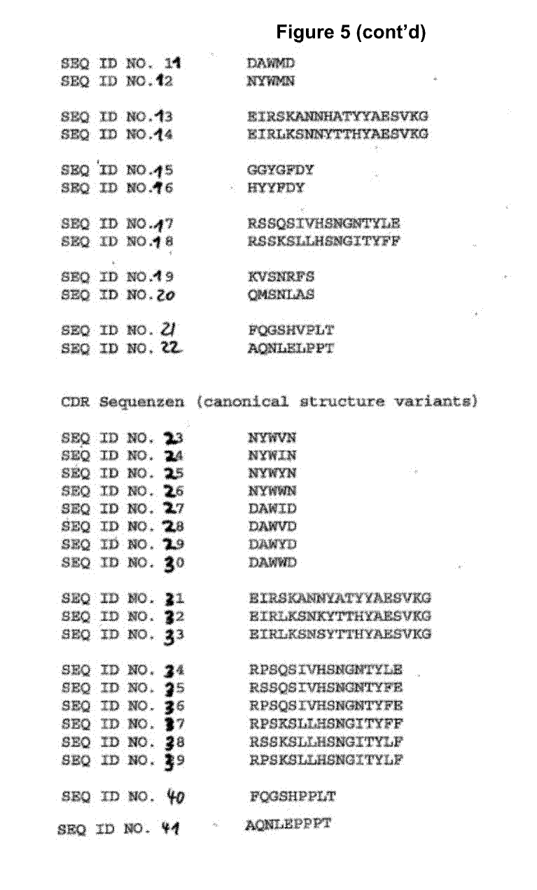

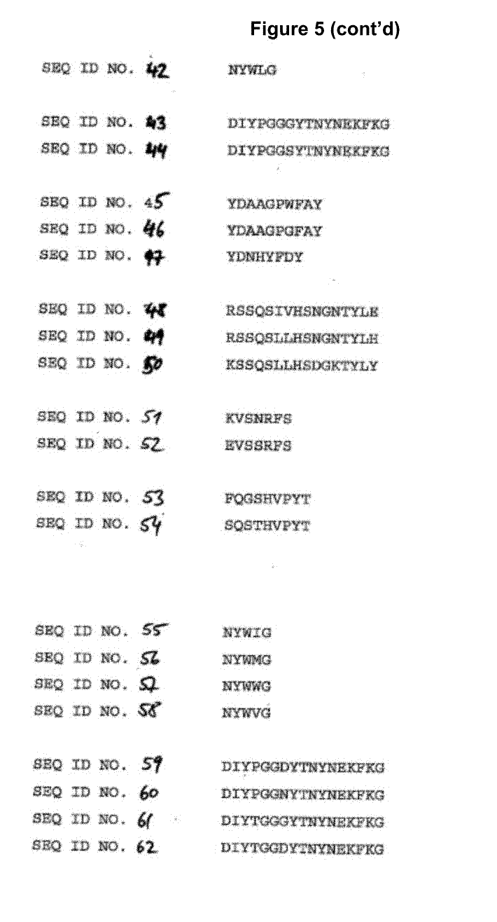

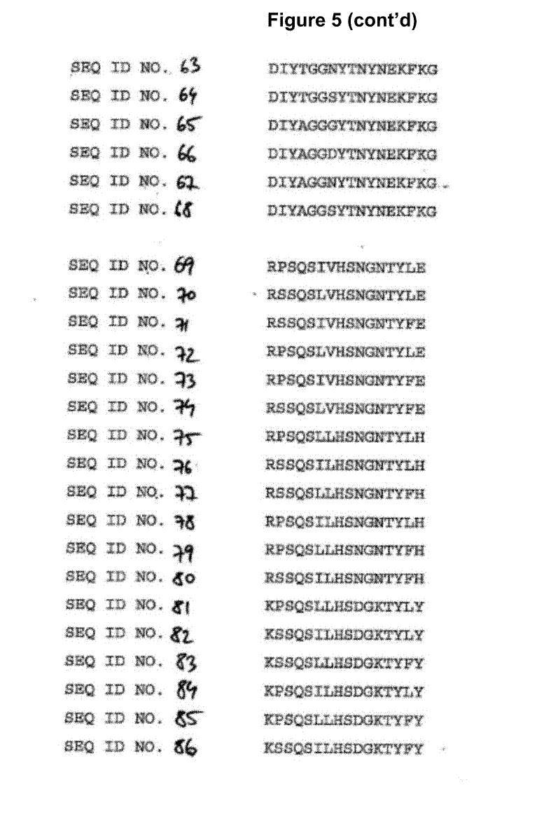

The antibody was labeled using the pHrodo™ Red Microscale Labeling Kit (life technologies). The conjugation was based on pHrodo™ succinimidyl ester which reacts with accessible amines of the antibody. Target cells were incubated at 4° C. (blue) or 37° C. (red) with indicated concentrations of antibody. After 4 h and 24 h target cells were assessed for pHrodo™ positivity using flow cytometry. The pH-sensitive dye indicates localization of antibody within acidic compartments of the target cell (see FIG. 4).

1. A method for improving the safety profile and/or efficacy of an antibody-drug-conjugate (ADC), comprising linking an antibody molecule to a drug in order to obtain said ADC, said antibody molecule being obtainable from a host cell selected to produce an antibody molecule composition having at least one of the following characteristics:
it comprises no detectable NeuGc; and/or
it has a galactosylation degree on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same antibody molecule isolated from ATCC No. CRL-9096 (CHOdhfr-) when expressed therein; and/or
it has an amount of GO structures on the total carbohydrate structures or on the carbohydrate structures at one particular glycosylation site of the antibody molecule of said antibody molecules in said antibody molecule composition which is at least 5% lower compared to the same amount of antibody molecules in at least one antibody molecule composition of the same antibody molecule isolated from ATCC No. CRL-9096 (CHOdhfr-) when expressed therein; and/or
it comprises no detectable terminal Galalpha1-3Gal; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein; and/or
it comprises at least one carbohydrate structure containing bisecting GlcNAc; and/or
it has a sialylation pattern which is altered compared to the sialylation pattern of at least one antibody molecule composition of the same antibody molecule isolated from ATCC No. CRL-9096 (CHOdhfr-) when expressed therein,
wherein improvement of said safety profile allows a reduction of the dose of said ADC to be administered to a patient in comparison to an ADC, the antibody part of which was preferably not obtained from a host cell having at least one of the glycosylation characteristics as defined above.

2. The method of claim 1, wherein said sialylation pattern is characterized by at least one of the following characteristics:
it comprises alpha2-6 linked NeuNAc; and/or
it has an increased sialylation degree with an amount of NeuNAc on the total carbohydrate structures or on the carbohydrate structures at one particular glycosylation site of the antibody molecule of the antibody molecules in said antibody molecule composition which is at least 15% higher compared to the same amount of antibody molecules in at least one antibody molecule composition of the same antibody molecule isolated from ATCC No. CRL-9096 (CHOdhfr-) when expressed therein, and/or
it comprises at least 20% more charged N-glycosidically linked carbohydrate chains of the total carbohydrate units or of at least one particular carbohydrate chain at a particular glycosylation site of the antibody molecule of the antibody molecules in said antibody molecule composition compared to the same amount of antibody molecules in at least one antibody molecule composition of the same antibody molecule isolated from ATCC No. CRL-9096 (CHOdhfr-) when expressed therein.

3. The method of claim 1 or 2, wherein said host cell is selected to produce an antibody, comprising
at least 10% carbohydrate structures of the total carbohydrate units or of at least one particular carbohydrate chain at a particular glycosylation site of the antibody molecule of the antibody molecules in said antibody molecule composition, lacking fucose; and/or
at least 2% carbohydrate structures of the total carbohydrate units or of at least one particular carbohydrate chain at a particular glycosylation site of the antibody molecule of the antibody molecules in said antibody molecule composition which contains bisecting GlcNAc; and/or
more than 35% G2 structures on the total carbohydrate structures or on the carbohydrate structures at one particular glycosylation site of the antibody molecule in said antibody molecule composition; and/or
it comprises less than 22% GO structures on the total carbohydrate structures or on the carbohydrate structures at one particular glycosylation site of the antibody molecule in said antibody molecule composition, and/or
wherein said host cell is selected to produce an antibody having the following characteristic glycosylation combinations:
(a)
it comprises no detectable NeuGc
it comprises no detectable Galalpha1-3Gal
it comprises a galactosylation degree on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein
it comprises bisecGlcNAc
it comprises an increased amount of sialic acid compared to a antibody composition of the same antibody molecule when expressed in the cell line ATCC No. CRL-9096 (CHOdhfr-) or compared to a sialylation deficient cell line such as DSM ACC2606 (NM-F9) and DSM ACC2605 (NM-D4); or
(b)
it comprises no detectable NeuGc
it comprises no detectable Galalphal -3Gal
it comprises a galactosylation degree on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein
it comprises bisecGlcNAc
it comprises 2-6 NeuNAc.

4. (canceled)

5. The method of claim 1, wherein said antibody
comprises no detectable NeuGc;
comprises a2,6-linked NeuNAc; and
has an increased sialylation degree with an amount of NeuNAc on the total carbohydrate structures or on the carbohydrate structures at one particular glycosylation site of the antibody molecule of said antibody molecules in said antibody molecule composition which is at least 15% higher compared to the same amount of antibody molecules in at least one antibody molecule composition of the same antibody molecule isolated from ATCC No. CRL-9096 (CHOdhfr-) when expressed therein.

6. The method of claim 1, wherein the antibody comprises at least 2%, preferably at least 5%, more preferably at least 10% and most preferably at least 15% carbohydrate structures of the total carbohydrate units or of at least one particular carbohydrate chain at a particular glycosylation site of an antibody molecule of the antibody molecules in said antibody molecule composition which contains bisecting GlcNAc.

7. The method of claim 1, wherein the antibody has an increased sialylation degree with an amount of NeuNAc on the total carbohydrate structures or on the carbohydrate structures at one particular glycosylation site of the antibody molecule of said antibody molecules in said antibody molecule composition which is at least 20%, preferably at least 30% higher compared to the same amount of antibody molecules in at least one antibody molecule composition of the same antibody molecule isolated from ATCC No. CRL-9096 (CHOdhfr-) when expressed therein.

8. The method of claim 1, wherein the antibody comprises at least 50%, preferably at least 60% and more preferably at least 70% carbohydrate structures of the total carbohydrate units or of at least one particular carbohydrate chain at a particular glycosylation site of an antibody molecule of the antibody molecules in said antibody molecule composition, lacking fucose.

9. The method of claim 1, wherein the antibody molecule composition comprises no detectable terminal Gala/pha1-3Gal.

10. The method of claim 1, wherein the antibody molecule has at least one of the following characteristics
it has a galactosylation degree of galactose, which is linked to GlcNAc, on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same antibody molecule isolated from ATCC No. CRL-9096 (CHOdhfr-) when expressed therein; and/or
it has an amount of GO structures on the total carbohydrate structures or on the carbohydrate structures at one particular glycosylation site of the antibody molecule of said antibody molecules in said antibody molecule composition which is at least 5% lower compared to the same amount of antibody molecules in at least one antibody molecule composition of the same antibody molecule isolated from ATCC No. CRL-9096 (CHOdhfr-) when expressed therein; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein.
it comprises at least 20% more charged N-glycosidically linked carbohydrate chains of the total carbohydrate units or of at least one particular carbohydrate chain at a particular glycosylation site of the antibody molecule of the antibody molecules in said antibody molecule composition compared to the same amount of antibody molecules in at least one antibody molecule composition of the same antibody molecule isolated from ATCC No. CRL-9096 (CHOdhfr-) when expressed therein, and/or
it comprises more than 35% G2 structures on the total carbohydrate structures or on the carbohydrate structures at one particular glycosylation site of an antibody molecule of the antibody molecules in said antibody composition; and/or
it comprises less than 22% GO structures on the total carbohydrate structures or on the carbohydrate structures at one particular glycosylation site of an antibody molecule of the antibody molecules in said antibody composition; and/or
it has an increased activity and/or increased yield compared to at least one antibody molecule composition of the same antibody molecule when expressed in the cell line ATCC No. CRL-9096 (CHOdhfr-); and/or
it has an improved homogeneity compared to at least one antibody molecule composition of the same antibody molecule when expressed in the cell line ATCC No. CRL-9096 (CHOdhfr-); and/or
it has an increased activity which is at least 10% higher than the activity of at least one antibody molecule composition from the same antibody molecule when expressed in the cell line ATCC No. CRL-9096 (CHOdhfr-);
and/or
it has an increased Fc-mediated cellular cytotoxicity which is at least 2 to 5 times higher than the Fc-mediated cellular cytotoxicity of at least one antibody molecule composition from the same antibody molecule when expressed in the cell line ATCC No. CRL-9096 (CHOdhfr-); and/or
it has an increased antigen mediated or Fc-mediated binding which is at least 50% higher than the binding of at least one antibody molecule composition from the same antibody molecule when expressed in the cell line ATCC No. CRL-9096 (CHOdhfr-); and/or - it has ADCC and/or CDC activity.

11. The method of claim 1, wherein said host cell is selected from the group consisting of NM-F9 [DSM ACC2606], NM-D4 [DSM ACC2605], GT-2X [DSM ACC2858], NM-H9, NM-E-2F9, NM-C-2F5, NM-H9D8 [DSM ACC2806], NM-H9D8-E6 [DSM ACC2807], NM-H9D8-E6Q12 [DSM ACC2856], GT-5s [DSM ACC 3078] or a cell or cell line derived therefrom.

12. The method of claim 1, wherein said ADC has enhanced Fc receptor (FcR) binding resulting in enhanced immunological effector functions of said ADC, said enhanced FcR binding is enhanced to the extent such that said ADC mediates enhanced immunological effector functions at doses at which essentially no immunological effector functions are mediated in comparison to said ADC without enhanced FcR binding.

13. The method of claim 12, wherein said enhanced FcR binding is enhanced Fc gamma receptor binding, particularly Fc gamma receptor Ill binding, and/or mediates enhanced antibody dependent cell cytotoxicity (ADCC).

14. An ADC obtainable by the method of claim 1 for use in a method of treatment of cancer, said method comprising administering said ADC at doses which are lower than doses for an ADC, the antibody part of which was preferably not obtained from a host cell having at least one of the glycosylation characteristics as defined above, whereby at said lower doses the Fc receptor (FcR) binding of said ADC is at least equal to or even improved in comparison to an ADC, the antibody part of which was preferably not obtained from a host cell having at least one of the glycosylation characteristics as defined above, wherein said antibody part is not directed to HER-2, BCMA, tenascin A2 and the A2 domain of tenascin A2 (TNC A2).

15. The ADC for the use of claim 14, wherein said ADC has enhanced Fc receptor (FcR) binding resulting in enhanced immunological effector functions of said ADC, said enhanced FcR binding is enhanced to the extent such that said ADC mediates enhanced immunological effector functions at doses at which essentially no immunological effector functions are mediated in comparison to said ADC without enhanced FcR binding.

16. The ADC for the use of claim 14, wherein said enhanced FcR binding is enhanced Fc gamma receptor binding, particularly Fc gamma receptor III binding, and/or mediates enhanced antibody dependent cell cytotoxicity (ADCC).

17. The ADC for the use of claim 14, wherein the antibody of said ADC is directed against a molecule on the surface of a cell, preferably a cancer cell, an immune cell, blood cell, or bone marrow cell, or a cell infected with a bacterium, virus or parasite.

18. The ADC for the use of claim 17, wherein said cancer is characterized by a solid tumor or is a blood-borne cancer, wherein the solid tumor is a breast cancer tumor.

19. (canceled)

20. The ADC for the use of claim 14, wherein said drug is a cytotoxin, such as a microtubule inhibitor or DNA-damaging agent, and/or
(A) wherein said antibody comprises
(i) a heavy chain variable region comprising a CDR1 having the amino acid sequence of SEQ ID NO: 1, a CDR2 having the amino acid sequence of SEQ ID NO: 2, and a CDR3 having the amino acid sequence of SEQ ID NO: 3; and
(ii) a light chain variable region comprising a CDR1 having the amino acid sequence of SEQ ID NO: 4, a CDR2 having the amino acid sequence of SEQ ID NO: 5, and a CDR3 having the amino acid sequence of SEQ ID NO: 6, and/or
(B) wherein said antibody comprises
(i) a heavy chain variable region comprising the amino acid sequence of SEQ ID NO: 7 or an amino acid sequence which is at least 80% identical thereto:
(ii) a light chain variable region comprising the amino acid sequence of SEQ ID NO: 8 or an amino acid sequence which is at least 80% identical thereto, and/or
(C) wherein said antibody comprises
(i) a heavy chain comprising the amino acid sequence of SEQ ID NO: 9 or an amino acid sequence which is at least 80% identical thereto;
(ii) a light chain variable region comprising the amino acid sequence of SEQ ID NO: 10 or an amino acid sequence which is at least 80% identical thereto.

21. to 23. (canceled)

24. An ex vivo use of a host cell for improving the safety profile and/or efficacy of an antibody-drug-conjugate (ADC), wherein said host cell is selected from the group consisting of NM-F9 [DSM ACC2606], NM-D4 [DSM ACC2605], GT-2X [DSM ACC2858], NM-H9, NM-E-2F9, NM-C-2F5, NM-H9D8 [DSM ACC2806], NM-H9D8-E6 [DSM ACC2807], NM-H9D8-E6Q12 [DSM ACC2856], GT-5s [DSM ACC 3078] or a cell or cell line derived therefrom.

25. The ex vivo use of a host cell according to claim 24, wherein an improvement is characterized by a reduction of the dose of an ADC to be administered to a patient in comparison to an ADC, the antibody part of which was preferably not obtained from a host cell having at least one of the glycosylation characteristics as defined below, whereby at said reduced dose the Fc receptor (FcR) binding of said ADC is at least equal to or even improved in comparison an ADC, the antibody part of which was preferably not obtained from a host cell having at least one of the glycosylation characteristics as defined below, wherein the glycosylation characteristic is selected from
it has a galactosylation degree on the total carbohydrate structures or on the carbohydrate structures at one particular glycosylation site of the antibody molecule of the antibody molecules in said antibody molecule composition, that is increased compared to the same amount of antibody molecules in at least one antibody molecule composition of the same antibody molecule isolated from ATCC No. CRL-9096 (CHOdhfr-) when expressed therein; and/or
it has an amount of G2 structures on the total carbohydrate structures or on the carbohydrate structures at one particular glycosylation site of the antibody molecule of said antibody molecules in said antibody molecule composition which is at least 5% higher compared to the same amount of antibody molecules in at least one antibody molecule composition of the same antibody molecule isolated from ATCC No. CRL-9096 (CHOdhfr-) when expressed therein; and/or
it has an amount of GO structures on the total carbohydrate structures or on the carbohydrate structures at one particular glycosylation site of the antibody molecule of said antibody molecules in said antibody molecule composition which is at least 5% lower compared to the same amount of antibody molecules in at least one antibody molecule composition of the same antibody molecule isolated from ATCC No. CRL-9096 (CHOdhfr-) when expressed therein; and/or
it comprises an amount of fucose on the total carbohydrate structures or on the carbohydrate structures at one particular glycosylation site of the antibody molecule of said antibody molecules in said antibody molecule composition which is at least 5% less compared to the same amount of antibody molecules in at least one antibody molecule composition of the same antibody molecule isolated from ATCC No. CRL-9096 (CHOdhfr-) when expressed therein.
